(19) United States
(12) Patent Application Publication
(10) Pub. No.: US 2011/0020239 A1
(43) Pub. Date: Jan. 27, 2011

(54) METHODS FOR IN VIVO IMAGING OF CELLS

(75) Inventors: Jeff Bulte, Fulton, MD (US); Bradley Powers Barnett, Baltimore, MD (US); Aravind Arepally, Baltimore, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 12/600,445

(22) PCT Filed: May 14, 2008
(86) PCT No.: PCT/US08/06380

§ 371 Date: Oct. 12, 2010

Related U.S. Application Data
(63) Continuation of application No. 61/001,159, filed on Oct. 31, 2007.
(60) Provisional application No. 60/930,150, filed on May 14, 2007.

(51) Int. Cl. A61K 49/00; C12N 5/00; C12N 13/00

(52) U.S. Cl. 424/9.6; 435/325; 424/9.1; 435/173.6

(57) ABSTRACT

The instant invention provides methods for the in vivo imaging of cells using one or more imaging modalities.

### RELATED APPLICATIONS

This application claims the benefit of U.S. Provisional Application No. 60/930,150, filed May 14, 2007, and U.S. Provisional Application No. 61/001,159, filed Oct. 31, 2007. The contents of each of the aforementioned applications is expressly incorporated herein by reference.

### GOVERNMENT SUPPORT

The following invention was supported at least in part by grants RO1 EB007825 and KO8 EB004348 from the National Institutes of Health. Accordingly, the government may have certain rights in the invention.

### BACKGROUND OF THE INVENTION

Many therapeutic strategies, such as stem cell transplantation, are based upon introducing exogenous living cells or tissues into a patient or host. A problem common to all therapeutic strategies involving administration of exogenous cells is identifying and monitoring the cells in the host. It is currently difficult or impossible to monitor the location of such cells or tissues in the host after administration with X-ray, ultrasound or MRI modalities. It may also be difficult to establish the survival of these cells in the host with such modalities. Ability to track cells with X-Ray and US modalities could potentially improve delivery strategies as commonly cells transplant procedures are clinically performed with x-ray or ultrasound guidance. Cellular therapy and diagnostics in humans would be advanced by a technique that can monitor cell fate non-invasively and repeatedly with one or more imaging modalities to assess the cellular biodistribution at a particular given time point.

### SUMMARY OF THE INVENTION

The instant invention is based, at least in part on the inventors discovery of novel methods for labeling cells in vivo and ex vivo for use in imaging in vivo. The invention provides methods and compositions for labeling cells and for using the labeled cells.

Accordingly, in one aspect, the instant invention provides methods of ex-vivo labeling of a cell for in vivo imaging by contacting a cell ex vivo with a labeling agent such that cell becomes labeled, thereby labeling a cell for in vivo imaging. In one embodiment, the cell is transplanted into a subject.

In another embodiment, the labeling agent is detectable by a modality selected from the group consisting of X-ray, CT, ultrasound, Raman, and magnetic resonance.

In another embodiment, the labeling agent is a multimode-detectable labeling agent, e.g., it is detectable by at least two modalities, e.g., such as X-ray, CT, ultrasound, Raman, and magnetic resonance.

In another embodiment the cell is a cell for use in cellular therapy, e.g., an immune cell, stem cell, progenitor cell, islet cell or other cell with regenerative properties.

In one embodiment, the labeling agent is a perfluorocarbon (PFC), e.g., perfluoro-15-crown-5-ether (PFCE), perfluorooctylbromide (PFOB). In another embodiment, the labeling agent is a colloidal metal particle, e.g., a colloidal gold or silver particle.

In another embodiment, the particle is a core-shell particle. In some embodiments, the shell of the core-shell particle is derivatized with functional groups for the conjugation of a bioactive molecule, e.g., a peptide or polypeptide such as an antibody of fragment thereof.

In another embodiment, the labeling agent is a gold-based agent, a silver-based agent, an iron-based agent, or a gadolinium-based agent. In related embodiments, the labeling agent is magnetic, paramagnetic or superparamagnetic.

In another embodiment, the cell is contacted with the labeling agent in the presence of a transfection agent. In another embodiment, the cell is electroporated in the presence of a labeling agent.

In another aspect, the invention provides methods of ex vivo labeling of a pancreatic β islet cell for in vivo imaging, by contacting the cell with a labeling agent ex vivo, thereby labeling the cell.

In one embodiment, the cell is transplanted into a subject.

In another embodiment, the labeling agent is detectable by a modality selected from the group consisting of X-ray, CT, ultrasound, Raman, and magnetic resonance.

In another embodiment, the labeling agent is a multimode-detectable labeling agent, e.g., it is detectable by at least two modalities, e.g., such as X-ray, CT, ultrasound, Raman, and magnetic resonance.

In another embodiment the cell is a cell for use in cellular therapy, e.g., an immune cell, stem cell, progenitor cell, islet cell or other cell with regenerative properties.

In one embodiment, the labeling agent is a perfluorocarbon (PFC), e.g., perfluoro-15-crown-5-ether (PFCE), perfluorooctylbromide (PFOB). In another embodiment, the labeling agent is a colloidal metal particle, e.g., a colloidal gold or silver particle.

In another embodiment, the particle is a core-shell particle. In some embodiments, the shell of the core-shell particle is derivatized with functional groups for the conjugation of a bioactive molecule, e.g., a peptide or polypeptide such as an antibody of fragment thereof.

In another embodiment, the labeling agent is a gold-based agent, a silver-based agent, an iron-based agent, or a gadolinium-based agent. In related embodiments, the labeling agent is magnetic, paramagnetic or superparamagnetic.

In another embodiment, the cell is contacted with the labeling agent in the presence of a transfection agent. In another embodiment, the cell is electroporated in the presence of a labeling agent.

In one embodiment, the β islet cell is transplanted into the kidney of a subject. In another embodiment, the labeled cell is imaged by CT and MR imaging. In further embodiments, the cell is imaged using ultrasound.

In another aspect, the instant invention provides methods for accurately transplanting cells into a subject by labeling cells with an imaging agent, guiding the injection of labeled cells using a first mode of detection, thereby accurately transplanting the cells.

In one embodiment, the imaging agent is a multimode-detectable imaging agent.

In another embodiment, the methods further comprise confirming the accuracy of injection using a second mode of detection and/or a third mode of detection.

In related embodiments, the first mode of detection is ultrasound, the second mode of detection is MR and the third mode of detection is CT.

In exemplary embodiments the imaging agent is a PFC or a colloidal metal particle. In one particular embodiment, the agent is PFOB.

In one embodiment, the cell is a β islet cell that is transplanted into a kidney.

In another aspect, the instant invention provides methods for accurately transplanting cells into a subject by labeling the cells with a multimodal imaging agent, guiding the injection of the labeled cells using ultrasound detection, thereby accurately transplanting the cells.

In one embodiment, the method further comprises confirming the accuracy of injection using MR and/or CT imaging.

In one embodiment, the agent is a PFC or a colloidal metal particle. In one exemplary embodiment, the agent is PFOB.

In one embodiment, the cell is a β islet cell and is transplanted into a kidney.

In another aspect, the instant invention provides methods of labeling a cell for in vivo imaging with a labeling agent by electroporating the cell in the presence of a metal containing particle, thereby labeling the cell with a multimodal labeling agent.

In one embodiment, the labeling agent, i.e., the metal containing particle, is a multimode detectable agent.

In another embodiment, the agent is a dextran based particle, e.g., an iron dextran particle, a gold dextran particle, or a silver dextran particle.

In one embodiment, the cell is a stem cell, e.g., a mesenchymal stem cell.

In another aspect, the instant invention provides methods of labeling a cell in vivo by administering to a subject a multimodal imaging agent, thereby labeling a cell in vivo.

In one embodiment, the multimodal imaging agent is specifically targeted to a specific cell type.

In one embodiment, the labeling agent is a perfluorocarbon (PFC), e.g., perfluoro-15-crown-5-ether (PFCE), perfluorooctylbromide (PFOB). In another embodiment, the labeling agent is a colloidal metal particle, e.g., a colloidal gold or silver particle.

In another embodiment, the particle is a core-shell particle. In some embodiments, the shell of the core-shell particle is derivatized with functional groups for the conjugation of a bioactive molecule, e.g., a peptide or polypeptide such as an antibody of fragment thereof. In one embodiment, the antibody or fragment thereof targets the labeling agent to a specific cell type, e.g., a cancer cell.

In another aspect, the instant invention provides ex vivo-labeled cells for multimodal in vivo imaging produced by the method set froth herein.

In one aspect, the invention provides methods of locating a cell comprising a multimode-detectable labeling agent in a subject comprising, obtaining two or more images of the subject or a portion thereof, overlaying the images, and analyzing the images to determine the location of the cell in the subject.

In one embodiment, the images are selected from X-ray, CT, ultrasound, Raman, and magnetic resonance images. In another embodiment, the analysis step is preformed using a computer program.

In another aspect, the instant invention provides methods of measuring the presence of a cell labeled with a fluorescent agent by labeling a cell with a fluorescent agent, irradiating a tissue comprising the cell with radiation, detecting a fluorescence emission spectrum of the fluorescent agent, thereby measuring the presence of a cell labeled with a fluorescent contrast agent.

In another aspect, the instant invention provides methods for determining if a cell contains a single or multiple contrast agents that produce a Raman spectra by a) labeling a cell with a raman reporting contrast agent by the method of any one disclosed herein by administering a contrast agent with antibody bound to the contrast agent so after systemic administration it binds to the antibody target; b) irradiating the tissue with a beam of infrared monochromatic light; c) obtaining the infrared Raman spectrum from the labeled cell d) comparing said infrared Raman spectrum so obtained from the labeled cells with the infrared Raman spectra correspondingly obtained from known samples of cells non containing contrast agent.

In another aspect the instant invention provides systems for monitoring the presence of a raman detectable agent in or on a cell using low-resolution Raman spectroscopy using a catheter having a first end and a second end with an excitation fiber extending therebetween, the excitation fiber suitable to transmit multi-mode radiation from the first end to the second end to irradiate a target region; a multi-mode laser coupled to the first end of the excitation fiber, the laser generates multi-mode radiation for irradiating the target region to produce a Raman spectrum consisting of scattered electromagnetic radiation; a low-resolution dispersion element positioned to receive and separate the scattered radiation into different wavelength components; a detection array, optically aligned with the dispersion element for detecting at least some of the wavelength components of the scattered light; and a processor for processing the data from the detector array to monitor a Raman detectable agent

In another aspect, the instant invention also provides kits comprising the cell produced by the methods described herein and instructions for use.

In one embodiment, the invention provides kit comprising reagents for labeling a cell for multimode-imaging and instructions for use.

In one embodiment, the invention provides kit comprising a cyropreserved cell that is labeled with a labeling agent and instructions for use. In one aspect, the labeling agent is a multimode-detectable labeling agent.

In a specific embodiment, the invention provides kits comprising β islet cells comprising a detectable label and instructions for transplanting the cell in to a subject. In one aspect, the labeling agent is a multimode-detectable labeling agent.

### DESCRIPTION OF THE DRAWINGS

FIG. 1 depicts rabbit MSCs labeled with Gold-dextran/PLL as described in examples reveals high efficiency of labeling. Cell nuclei labeled with DAPI (blue) and the dextran component of Golddextran labeled in red with anti-dextran antibody as described in examples.

FIG. 2 depicts rabbit MSCs labeled with Gold-dextran via electroporation as described in examples reveals high efficiency of labeling. Cell nuclei labeled with DAPI (blue) and the dextran component of Gold-dextran labeled in red with anti-dextran antibody as described in examples.

FIG. 3 depicts high power of Rabbit MSCs labeled with Gold-dextran via electroporation as described in examples reveals high efficiency of labeling. Cell nuclei labeled with DAPI (blue) and the dextran component of Gold-dextran labeled in red with anti-dextran antibody as described in examples.

FIG. 4 depicts rabbit MSCs labeled with Gold-dextran via tat-peptide as described in examples reveals high efficiency of labeling. Cell nuclei labeled with DAPI (blue) and the dextran component of Gold-dextran labeled in red with anti-dextran antibody as described in examples.

FIG. 5 depicts closeup of Rabbit MSCs labeled with Gold-dextran via tat peptide as described in examples reveals high efficiency of labeling. Cell nuclei labeled with DAPI (blue) and the dextran component of Gold-dextran labeled in red with anti-dextran antibody as described in examples.

FIG. 6 depicts rabbit MSCs labeled with Gold-dextran via protamine sulfate as described in examples reveals high efficiency of labeling. Cell nuclei labeled with DAPI (blue) and the dextran component of Gold-dextran labeled in red with anti-dextran antibody as described in examples.

FIG. 7 depicts rabbits MSCs labeled with gold-dextran/pll as described in examples and suspended as approximated point sources in a gelatin phantom at cell concentration of A) 1×103, B) 1×104 C) 1×105, D) 1×106 cells. Phantom was imaged on CorE 64 Multislice CT. Effective slice thickness was 0.6 mm with. A reconstruction increment of 0.3 mm was applied.

FIG. 8 depicts rabbits MSCs labeled with gold-dextran/pll as described in examples and suspended as approximated point sources in a gelatin phantom at cell concentration of 1×104, 1×105 and 1×106 cells. Phantom was imaged on CorE 64 Multislice CT and 3D reconstruction was performed on AMIRA software.

FIG. 9 depicts rabbits MSCs labeled with gold-dextran/pll as described in examples and suspended as approximated point sources in a gelatin phantom at cell concentration of A) 1×104 B) 1×105, C) 1×106 cells. Phantom was imaged on with standard clinical grade portable US. Sonography was performed with a L25E 13-6 MhZ probe on a Micromaxx US system (Sonsite). Grayscale imaging was performed with a center probe frequency of 6.00 MHz, a dynamic range of 55 dB, and a persistence setting of two.

FIGS. 10A-D depict a viability assessment of labeled islet cells. (A) MTS assay of PFOB, PFPE and Feridex labeled and unlabeled human islets. (B) Percent survival of islets on day 14 post labeling. (c) Glucose responsiveness stimulation index (c-peptide secretion at 8 mM glucose/c-peptide secretion at 6 mM glucose) of islets on day 14. (D) Percent survival and glucose responsiveness stimulation index of islets after 1, 7, and 14 days.

FIGS. 11A-C depict labeled islet cells. (A) 10, 50, 100 and 200 islets labeled with PFPE. ¹⁹F MRI/¹H MRI overlay of mouse kidney with PFOB labeled islets. (B) single plane. (C) 3-d reconstruction.

FIGS. 12A-D depicts images of islet cells. (A) fluorescent microscopy of labeled islet with PFPE/rhodamine, (B) CT of two FPOB labeled islet clusters in a phantom, (C and D) Single pass and 3-d reconstruction CT of a mouse with FPOB labeled islets in vivo, respectively.

FIGS. 13A-D depict Feridex labeling and MR imaging of human pancreatic islet cells. (A) staining with anti-dextan FITC for feridex and DAPI for nuclei. (B) Prussina Blue (Fe3+ specific) staining of Feridex labeled human islets. Human islets were embedded in a 2% gelatin phantom at a density of 50 islets/ml gel, (C) using conventional T2*-weighted images, individual islets can be identified as hypontensities. (D) close up of outlined area in (C).

FIGS. 14A-B depicts a Raman spectra of a control containing only gold-dextran particles, and stem cells labeled with gold-dextran particles.

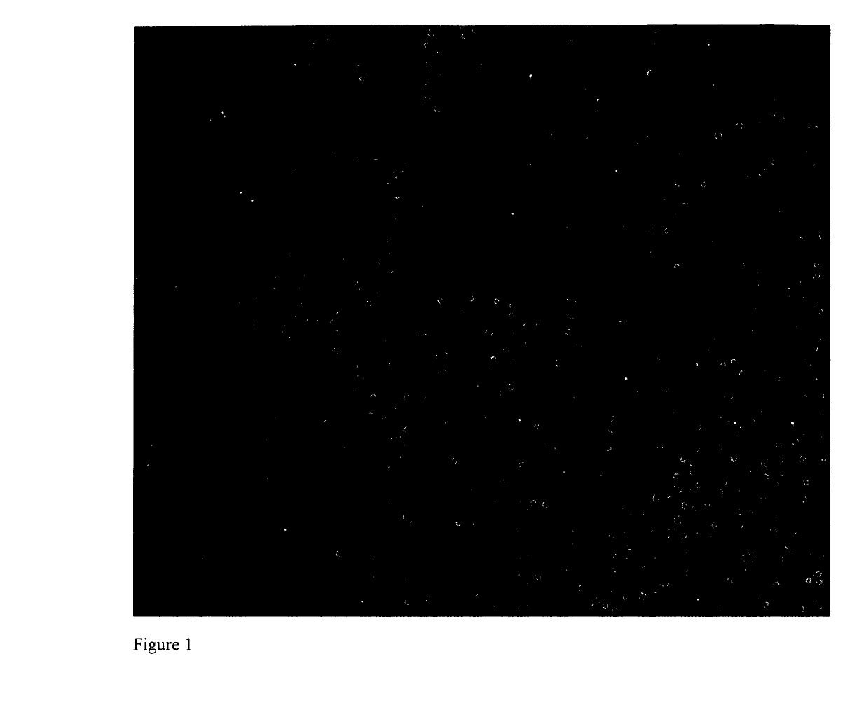

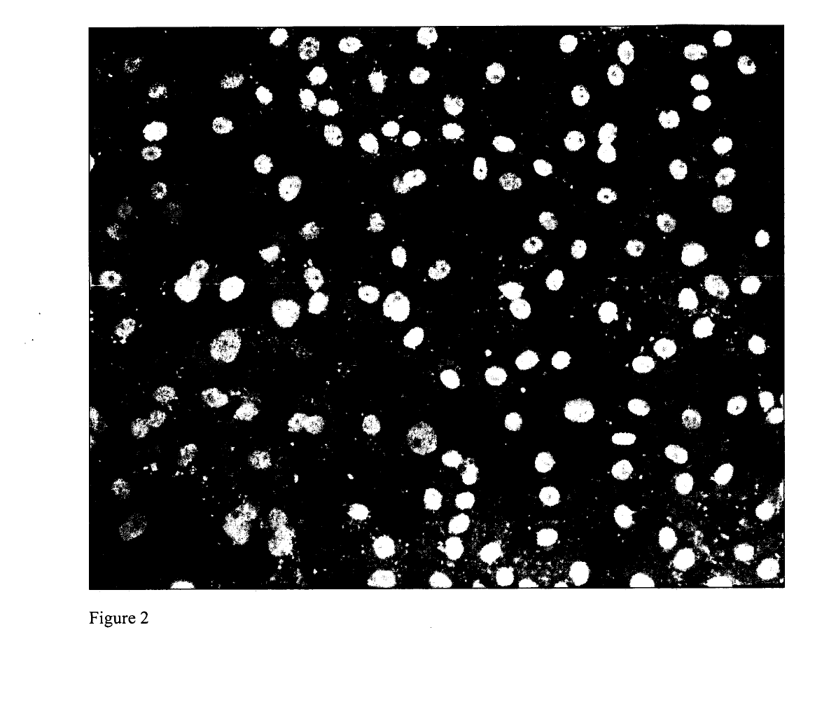

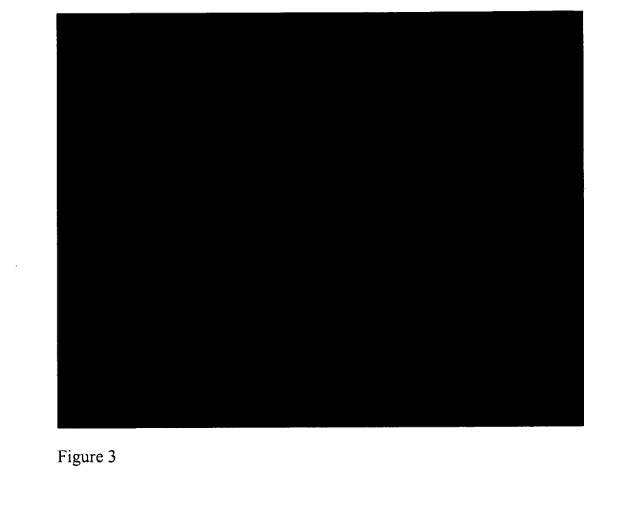

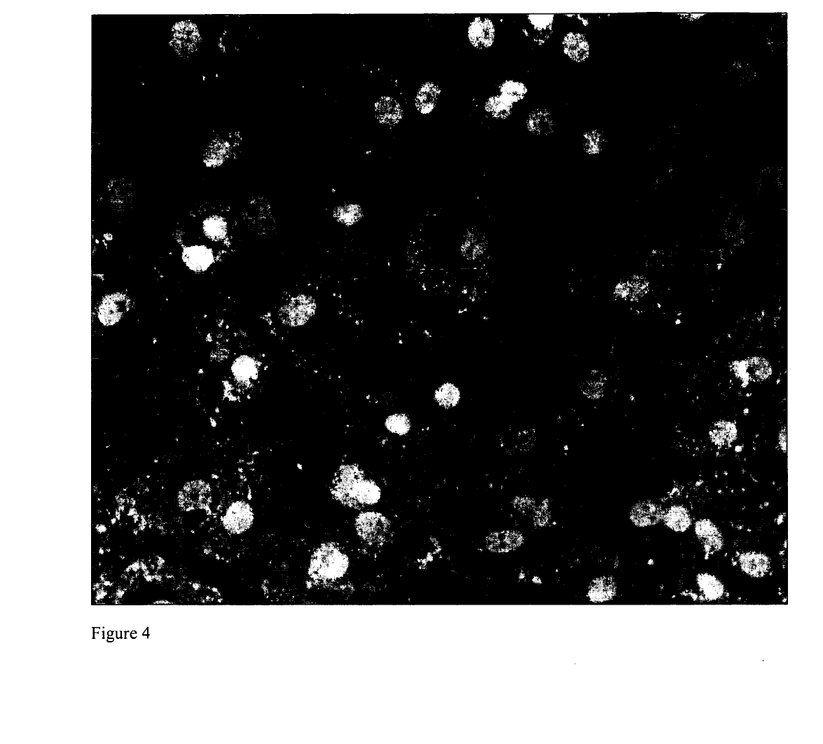

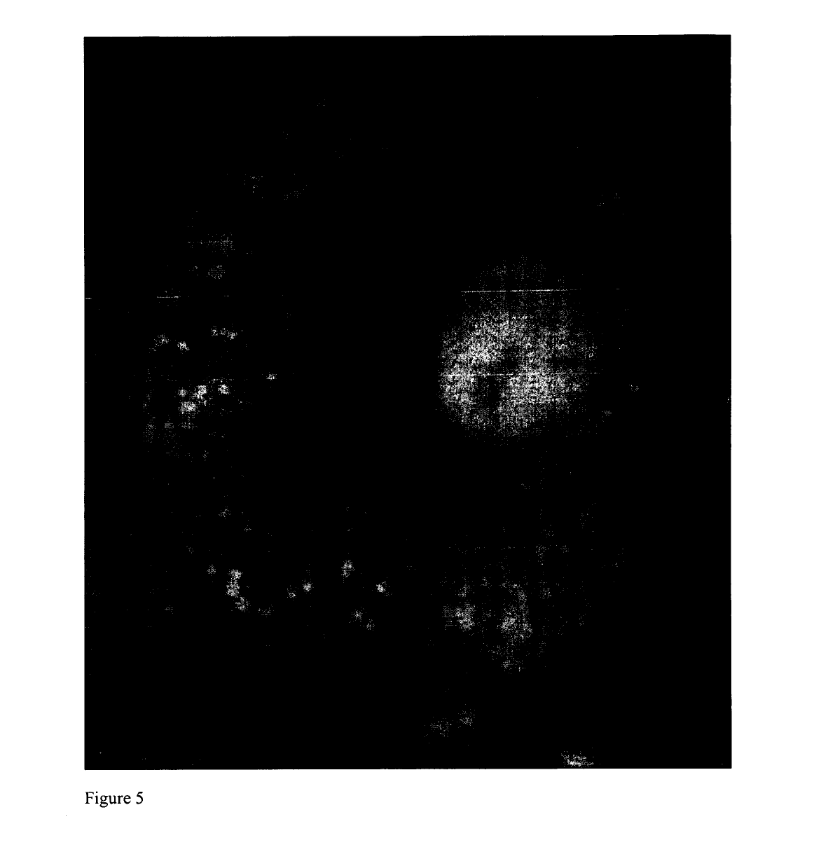

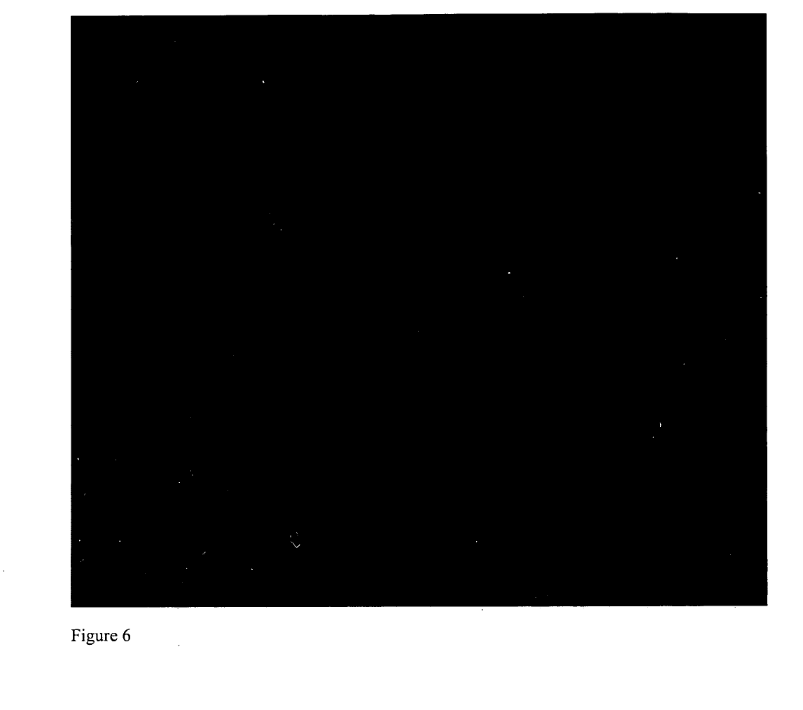

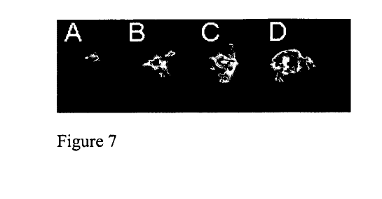

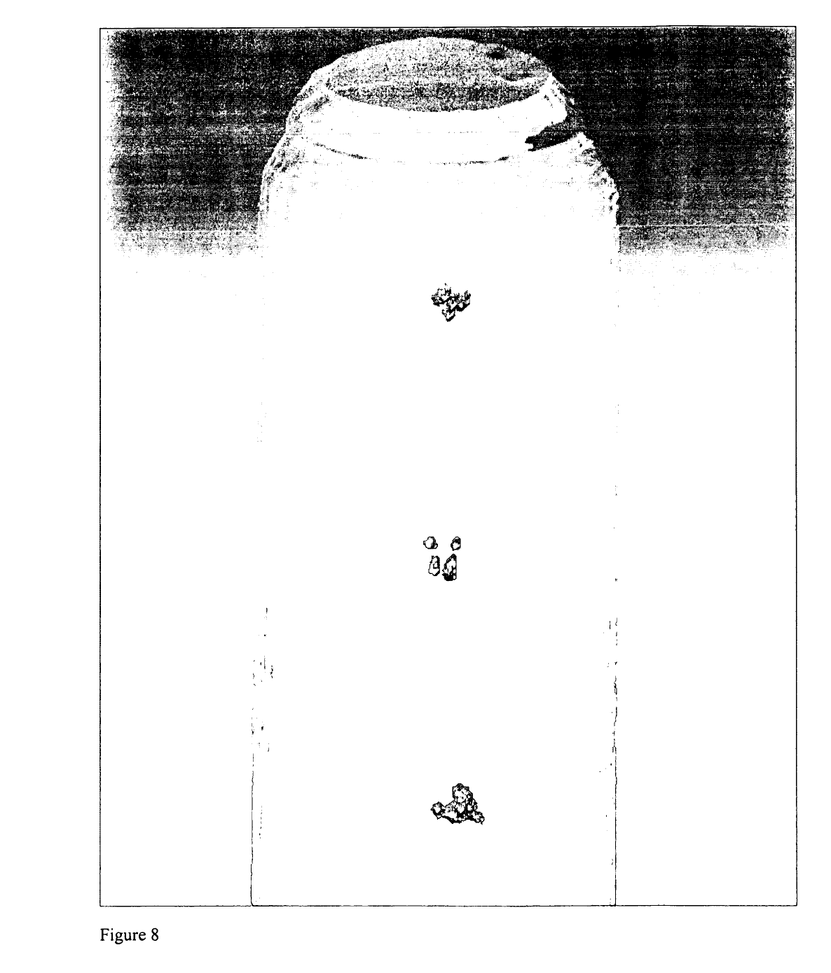

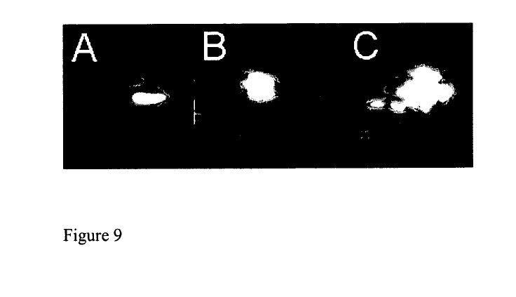

### DETAILED DESCRIPTION OF THE INVENTION

The instant invention is based on the inventors discovery of novel methods for labeling cells for detection in vivo. In one embodiment, the cells are labeled using a multimode-detectable label such as those described herein. The methods of the invention allow for in vivo or ex vivo labeling of cells for detection of the cells in vivo.

A “contrast agent,” as used herein, refers to a compound employed to improve the visibility of a cell in an image, e.g., a CT or MRI image. The term contrast agent is also referred to herein as an imaging agent or a detectable labeling. Contrast agents can be internalized by a cell or attached to a cell by, for example, an antibody.

“Particles” include, for example, liposomes, micelles, bubbles containing gas and/or gas precursors, lipoproteins, halocarbon, nanoparticle and/or hydrocarbon nanoparticles, halocarbon and/or hydrocarbon emulsion droplets, hollow and/or porous particles and/or solid nanoparticles. The particles themselves may be of various physical states, including solid particles, solid particles coated with liquid, liquid particles coated with liquid, and gas particles coated with solid or liquid. Various particles useful in the invention have been described in the art as well as means for coupling targeting components to those particles in the active composition. Such particles are described, for example, in U.S. Pat. Nos. 6,548,046; 6,821,506; 5,149,319; 5,542,935; 5,585,112; 5,149,319; 5,922,304; and European publication 727,225, all incorporated herein by reference with respect to the structure of the particles. These documents are merely exemplary and not all-inclusive of the various kinds of particulate vehicles that are useful in the invention. While nanoparticles are generally described herein, it is understood that the embodiments of the invention are not limited to nanoparticles, and that the compositions and methods described herein are similarly useful for other types of particles.

As used herein, the term “subject” means any organism. The term need not refer exclusively to a human being, one example of a subject, but can also refer to animals such as mice, rats, dogs, poultry, and even tissue cultures. The methods disclosed herein are particularly useful in warm-blooded vertebrates, e.g., mammals.

As used herein, “multimodal” means at least two imaging modes which differ in their spectral bands of illumination or their spectral bands of detection, or both. The present invention provides multimodal detection agents that, by virtue of their fluorescent, radio-opaque, and/or paramagnetic properties, function as contrast agents using one or more imaging modalities. These multifunctional detection agents aid in the detection and/or localization of cells. In some embodiments, the multimodal detection agents of the invention and methods of using the same allow for precise, direct, real-time visualization of cells. For example, multimodal refers to two or more of ultrasound, CT, magnetic resonance, PET, X-ray and Raman modalities.

The term “cell” is understood to mean embryonic, fetal, pediatric, or adult cells or tissues, including but not limited to, stem cells, precursors cells, and progenitor cells. In one embodiment, the cell is an islet cell. It is also understood that the term “cells” encompasses virus particles and bacteria.

Exemplary cells include immune cell, stem cell, progenitor cell, islet cell, bone marrow cells, hematopoietic cells, tumor cells, lymphocytes, leukocytes, granulocytes, hepatocytes, monocytes, macrophages, fibroblasts, neural cells, mesenchymal stem cells, neural stem cells, or other cell with regenerative properties and combinations thereof.

Imaging Agents

The invention provides methods of labeling cells using one or more labeling agents. In certain embodiments the labeling agent is a multimode-detectable agent.

In one embodiment the methods of the invention use magnetic particles in the methods of imaging cells. The magnetic particles include a metal oxide particle and a coating material that is in contact with the surface of the metal oxide particle.

The metal of the metal particle may include transition or lanthanide metals. Illustrative transition or lanthanide metals include iron, cobalt, gadolinium, europium and manganese. The magnetic-responsive metal oxide particles may be paramagnetic, ferrimagnetic, superparamagnetic or anti-ferromagnetic.

The coating material may be in contact with the metal oxide particle surface via any type of chemical bonding and/or physical attractive force such as, for example, covalent bonding, ionic bonding, hydrogen bonding, colloidal mixtures or complexing.

Illustrative coating materials include polysaccharides, polyvinyl alcohols, polyacrylates, polystyrenes, and mixtures and copolymers thereof. According to a particular embodiment the coating material is a polysaccharide such as, for example, starch, cellulose, glycogen, dextran, aminodextran and derivatives thereof.

According to a particular embodiment the metal particle is a metal oxide particle, e.g., an iron oxide, especially a superparamagnetic iron oxide. Superparamagnetic iron oxides are (on a millimolar metal basis) the most MR-sensitive tracers currently available.

Superparamagnetic particles possess a large ferrimagnetic moment that, because of the small crystal size, is free to align with an applied magnetic field (i.e., there is no hysteresis). The aligned magnetization then creates microscopic field gradients that dephase nearby protons and shorten the T2 NMR relaxation time, over and beyond the usual dipole-dipole relaxation mechanism that affects both T1 and T2 relaxation times.

Examples of superparamagnetic iron oxides include MION-46L (available from Harvard Medical School), Feridex (commercially available from Berlex Laboratories, Inc. under license from Advanced Magnetic, Inc), Endorem ferumoxides (commercially available from Guerbet Group), Clariscan (commercially available from Nycomed Amersham), Resovist (commercially available from Schering AG), Combidex (commercially available from Advanced Magnetics), and Sinerem2) (commercially available from Guerbet Group under license from Advanced Magnetics).

MION-46L is a dextran-coated nanoparticle with a superparamagnetic maghemite- or magnetite-like inverse spinel core structure. Feridex is a FDA-approved aqueous colloid of superparamagnetic iron oxide associated with dextran for intravenous administration.

Resovist consists of superparamagnetic iron oxide particles coated with carboxydextran.

In other embodiments, the methods of the invention use Chemical Exchange Saturation Transfer (CEST) Agents or PARACEST agents.

As described above, the coated metal particles can be used as magnetic probes. The magnetic probes can achieve a high degree of intracellular magnetic labeling that is non-specific (i.e., not dependent on targeted membrane receptor binding) and that can be used on virtually any mammalian cell. The magnetic probe could be used to label cells in vivo or ex vivo, for example, as an MR contrast agent, magnetic guidance of cells, ultrasound imaging.

Chemical modification of the coating on the coated metal oxide particles is not required. Furthermore, the mixing may be accomplished without the presence of an organic solvent. The amount of coated metal oxide particles mixed optionally with a transfection agent should be sufficient to provide uptake of the metal particles by the cell.

One particular embodiment includes labeling living cells with the metal particle to render the cells labeled for imaging. Such magnetically labeled cells may be prepared as described herein. For example, a cell of interest can be cultured in a standard media that includes the iron oxide and a transfection agent at a dose ranging from about 5 to about 100 Fg Fe/ml, more particularly about 5 to about 25 jug Fe/ml. Alternatively, the metal particle transfection agent mixture can be injected into tumors and other areas to label cells in situ or by injecting into blood vessels, ventricles or other brain or body cavities. The magnetically labeled cells may be exogenously applied to a host and monitored within the host using MRI and/or other imaging modalities. For example, such cells may be injected, tranasplanted or otherwise applied to the host.

Other useful metals also include isotopes of those metals possessing paramagnetism which produce water relaxation properties useful for generating images with magnetic resonance imaging (MRI) devices. Suitable relaxivity metals include, but are not limited to, Mn, Cr, Fe, Gd, Eu, Dy, Ho, Cu, Co, Ni, Sm, Tb, Er, Tm, and Yb. Appropriate chelation ligands to coordinate MR relaxivity metals can be readily incorporated into the peptide complexes of this invention by the methods previously described for radionuclides. Such chelation ligands can include, but are not limited to, DTPA, EDTA, DOTA, TETA, EHPG, HBED, ENBPI, ENBPA, and other macrocycles known to those skilled in the art (Stark and Bradley, Magnetic Resonance Imaging, C. V. Mosby Co., St Louis, 1988, pp 1516).

The invention also provides methods of using perfluorocarbons (PFCs). Representative perfluorocarbons include bis(F-alkyl)ethanes such as F-44E, i-F-i36E, and F-66E; cyclic fluorocarbons, such as F-decalin, perfluorodecalin or “FDC), F-adamantane (“FA”), F-methyladamantane (“FMA”), F-1,3-dimethyladamantane (“FDMA”), F-di- or F-trimethylbicyclo[3,3,1]nonane (“nonane”); perfluorinated amines, such as F-tripropylamine (“FTPA”) and F-tri-butylamine (“FTBA”), F-4-methyloctahydroquinolizine (“FMOQ”), F-n-methyl-decahydroisoquinoline (“FMIQ”), F-n-methyldecahydroquinoline (“FHQ”), F-n-cyclohexylpurrolidine (“FCHP”) and F-2-butyltetrahydrofuran (“FC-75” or “RM101”). Brominated perfluorocarbons include 1-bromo-heptadecafluoro-octane (sometimes designated perfluorooctylbromide or “PFOB”), 1-bromopenta-decafluoroheptane, and I-bromotridecafluorohexane (sometimes known as perfluorohexylbromide or “PFHB”). PFOB is a preferred lableing agent for use in the methods of the invention. Other brominated fluorocarbons are disclosed in U.S. Pat. No. 3,975,512. Other suitable perfluorocarbons are mentioned in EP 908 178 A1. Cobalt Nanoparticles, Iron Oxide Nanopowder, Niobium Oxide Nanopowder, Thulium Nanoparticles, Cobalt Oxide Nanopowder, Lanthanum Nanoparticles, Palladium Nanoparticles, Tin Nanoparticles, Aluminum Oxide Nanopowder, Copper Nanoparticles, Lanthanum Oxide Nanopowder, Platinum Nanoparticles, Tin Oxide Nanopowder, Antimony Nanoparticles, Copper Oxide Nanopowder, Praseodymium Nanoparticles, Titanium Carbide Nanoparticles, Antimony Oxide Nanopowder, Dysprosium Nanoparticles, Lithium Manganese Oxide Nanoparticles, Praseodymium Oxide Nanopowder, Titanium Nanoparticles, Antimony Tin Oxide (ATO) Nanoparticles, Dysprosium Oxide Nanopowder, Lithium Nanoparticles, Rhenium Nanoparticles, Titanium Nitride Nanoparticles, Barium Titanate Nanoparticles, Erbium Nanoparticles, Lithium Titanate Nanoparticles, Ruthenium Nanoparticles, Titanium Oxide Nanopowder, Beryllium Nanoparticles, Erbium Oxide Nanopowder, Lithium Vanadate Nanoparticles, Samarium Nanoparticles, Tungsten Carbide Nanoparticles, Bismuth Oxide Nanopowder, Europium Nanoparticles, Lutetium Nanoparticles, Samarium Oxide Nanopowder, Tungsten Nanoparticles, Boron Carbide Nanoparticles, Europium Oxide Nanopowder, Magnesium Nanoparticles, Silicon Carbide Nanoparticles, Tungsten Oxide Nanopowder, Boron Nitride Nanoparticles, Gadolinium Nanoparticles, Magnesium Oxide Nanopowder, Silicon Nanoparticles, Vanadium Oxide Nanopowder, Calcium Carbonate Nanoparticles, Gadolinium Oxide Nanopowder, Manganese Nanoparticles, Silicon Nanotubes, Ytterbium Nanoparticles, Calcium Chloride Nanoparticles, Gold Nanoparticles, Manganese Oxide Nanopowder, Silicon Nitride Nanoparticles, Yttria stabilized Zirconia, Calcium Oxide Nanopowder, Hafnium Oxide Nanopowder, Molybdenum Nanoparticles, Silicon Oxide Nanopowder, Yttrium Nanoparticles, Calcium Phosphate Nanoparticles, Holmium Nanoparticles, Molybdenum Oxide Nanopowder, Silver Nanoparticles, Zinc Oxide Nanopowder, Carbon Nanohorns, Indium Nanoparticles, Neodymium Nanoparticles, Strontium Carbonate Nanoparticles, Zirconium Nanoparticles, Carbon Nanoparticles Indium Oxide Nanopowder, Neodymium Oxide Nanopowder, Strontium Titanate Nanoparticles, Zirconium Oxide Nanopowder, Carbon Nanotubes, Iridium Nanoparticles, Nickel Nanoparticles, Tantalum Nanoparticles, Cerium Nanoparticles, Iron Cobalt Nanopowder, Nickel Oxide Nanopowder, Tantalum Oxide Nanopowder, Cerium Oxide Nanopowder, Iron Nanoparticles, Nickel Titanium Nanopowder, Terbium Nanoparticles, Chromium Oxide Nanopowder, Iron Nickel Nanopowder, Niobium Nanoparticles, Terbium Oxide Nanopowder, Carbon 60 fullerenes, Carbon 70 fullerens and Carbon 85 fullerenes, single wall carbon nanotubes, multi-wall carbon nanotubes, carbon nanofibers.

In one embodiment, the labeling agent is a radiopaque agent. As disclosed therein, monobromo, and dibromo perfluorocarbons, including both aliphatic and cyclic compounds, exhibit radiopaque properties which make such brominated perfluorocarbons useful.

The methods of the instant invention can use fluorescent labeling agents. Numerous fluorescent agents are available for use in the methods of the invention. Exemplary fluorescent labeling agents include, Rhodamine 101, Nile Red, Nileblue A, Fluorescein, Sulforhodamine B, Sulforhodamine G, PdTFPP, DiA, 5(6)-Carboxyfluorescein, 2, 7 [symbol] Dichlorofluorescein, 1,1\-Diethyl-4,4\-carbocyanine iodide, 3,3-Diethylthiadicarbocyanine iodide, Lucifer Yellow CH Dilitium salt 5(6)-Carboxytetramethylrhodamine B, N,N-Bis(2,4,6-trimethylphenyl)-3,4:9,10-perylenebis(dicarboximide, Rhodamine B, 2-Di-1-ASP, Dichlorotris(1,10-phenanthroline)ruthenium(II), Tris(4,7-diphenyl-1,10-phenanthroline)ruthenium(II) TMS, Tris(4,4-diphenyl-2,2-bipyridine)ruthenium(II), chloride, Resorufin, Ethyl Eosin, Ethyl Eosin, Coumarin 6, Rhodamine 6G, 8-Benzyloxy-5,7-diphenylquinoline, 8-Benzyloxy-5,7-diphenylquinoline (protonated), DY-500XL, DY-554, DY-633, DY-615, DY-590, DY-650, DY-490XL, DY-520XL, DY-485XL, DY-480XL, DY-555, DY-590, DY-630, DY-631, DY-635, DY-636, DY-647, DY-651, DY-656, DY-673, DY-675, DY-676, DY-680, DY-681, DY-700, DY-701, DY-730, DY-731, DY-750, DY-751, DY-776, DY-782, EVOblue-30, Adams Apple Red 680, Adirondack Green 520, Birch Yellow 580, Catskill Green 540 Fort Orange 600, Hemo Red 720, Hops Yellow 560, Lake Placid 490, Maple Red-Orange 620, Snake-Eye Red 900, QD525, QD565, QD585, QD605, QD655, QD705, QD800, ATTO 465, ATTO 425, ATTO 488, ATTO 495, ATTO 520, ATTO 550, ATTO 565, ATTO 590, ATTO 610, ATTO 620, ATTO 635, ATTO 647, ATTO 655, ATTO 680, ATTO 700, Alexa Fluor 350, Alexa Fluor 430, Alexa Fluor 480, Alexa Fluor 633, 5-FAM, DyLight 549 5-TAMRA, 6-HEX, 6-carboxyrhodamine 6G, 6-JOE, 6-TET, BOBO-1, BOBO-3, POPO-1, POPO-3, TOTO-1, TOTO-3, YOYO-1, YOYO-3, aminomethylcoumarin, APC, BCECF, Amplex Gold (product), dichlorofluorescein, TO-PRO-1, TO-PRO-3, SYTO 11, SYTO 13, SYTO 17, SYTO 45, PO-PRO-1, PO-PRO-3, propidium iodide, Pro-Q Diamond, Pro-Q Emerald, quinine, resorufin, rhod-2, rhodamine 110, rhodamine 123, Rhodamine Green, YO-PRO-1, YO-PRO-3, SYTOX Blue, SYTOX Green, SYTOX Orange, Rhodamine Red-X rhodamine, Rhodol Green, R-phycoerythrin, SBFI, Sodium Green sulforhodamine 101, SYBR Green I, SYPRO Ruby, tetramethylrhodamine, Texas Red-X, X-rhod-1, Alexa Fluor 500, Alexa Fluor 514, Alexa Fluor 610, Alexa Fluor 635 Calcein red-orange, Carboxynaphthofluorescein, DiIC18(3, ELF 97, Ethidium bromide, Alexa Fluor 532, Alexa Fluor 546, Alexa Fluor 555, Alexa Fluor 568, Alexa Fluor 594, Alexa Fluor 610-R-PE, Alexa Fluor 647, Alexa Fluor 647-R-PE Alexa Fluor 660, Alexa Fluor 680, Alexa Fluor 680-APC, Alexa Fluor 680-R-PE Alexa Fluor 700, Alexa Fluor 750, FITC, Fluo-3, Fluo-4, fluoro-emerald, FM 1-43 FM 4-64, Hoechst 33258, JC-1, JOJO-1, LOLO-1, lucifer yellow CH, LysoSensor Blue DND-192, LysoSensor Green DND-153, YoYo-1 ssDNA, YoYo-1 dsDNA, YoYo-1, Yakima Yellow, tdTomato, Tb (Soini), SYTO RNASelect, SYTO RNASelect, Calcofluor white 2MR, DAPI, DDAO, Deep Purple, Diversa Cyan-FP, Diversa Green-FP, Dragon Green Envy Green, Ethidium bromide, Ethyl Nile Blue A, Eu (Soini), Eu203 nanoparticles, EvaGreen, mBanana, mCherry, Methylene Blue, Methylene Blue, Flash Red EX, mHoneyDew, mOrange, mPlum, mRaspberry, mRFP1.2 (Wang), mStrawberry (Shaner), mTangerine (Shaner), Pacific Orange, Plum Purple, Pontamine fast scarlet 4B, Surf Green EX, Suncoast Yellow, Cresyl Violet Perchlorate, DyLight 488 Allophycocyanin, Coumarin 6, C-Phycocyanin, CryptoLight CF1, CryptoLight CF2, CryptoLight CF3, CryptoLight CF4, CryptoLight CF5, CryptoLight CF6, R-phycoerythrin, SensiLight PBXL-1, SensiLight PBXL-3, Spectrum Aqua, Spectrum Blue, Spectrum Fred, Spectrum Gold, Spectrum Green, Spectrum Orange, Spectrum Red, 1,4-Diphenylbutadiene, 1,2-Diphenylacetylene, 1,4-Diphenylbutadiyne 1,6-Diphenylhexatriene, Ir(Cn)₂(acac), 7-Methoxycoumarin-4-Acetic Acid 9,10-Bis(Phenylethynyl)Anthracene, 9,10-Diphenylanthracene, Acridine Orange Acridine Yellow, Anthracene, Auramine O, Benzene, Cy3B Biphenyl, C3-Indocyanine C3-Indocyanine, C3-Oxacyanine, C3-Thiacyanine Dye (EtOH), C3-Thiacyanine Dye (PrOH), C5-Indocyanine, C5-Oxacyanine, C5-Thiacyanine, C7-Indocyanine, C7-Oxacyanine, Coumarin 1, Dye-33, Dye-28, Dye-45, Cy3, DRAQ5, Ethyl-p-Dimethylaminobenzoate, Cy3.5, Cy2, CBQCA, Oregon Green 514, Oregon Green 488, nile red, nile blue, NeuroTrace 500525, NBD-X, monobromobimane, MitoTracker Red CMXRos, MitoTracker Orange CMTMRos, MitoTracker Green FM, Marina Blue, Magnesium Orange, Magnesium Green, LysoTracker Red DND-99, LysoTracker Green DND-26, LysoTracker Blue DND-22, LysoSensor YellowBlue DND-160, LysoSensor Green DND-153, LysoSensor Blue DND-192, lucifer yellow CH, JC-1, indo-1, fora-2, Fura Red, Coumarin 343, Cy3Cy5 ET, Cy5.5, Cy5, Cy7, CypHer5, Coumarin 30, Coumarin 314, ECF, ECL Plus, PA-GFP (post-activation), PA-GFP (pre-activation), WEGFP (post-activation), CHOxAsH-CCXXCC, FlAsH-CCXXCC, ReAsH-CCXXCC, NIR1, NIR2, NIR3, NIR4, NIR820, SNIR1, SNIR2, SNIR4, AmCyan1, AsRed2, Azami Green monomeric, Azami Green, CFP (Campbell Tsien 2003), Citrine (Campbell Tsien 2003), DsRed, DsRed, DsRed Dimer2 (Campbell Tsien 2003), DsRed-Express T1, EBFP (Patterson 2001), ECFP (Patterson 2001), EGFP (Campbell Tsien 2003), EGFP (Patterson 2001), Eosin Y, Fluorescein, Fluorescein-Dibase, Hoechst-33258, Hoechst-33258, Kaede Green, Magnesium Octaethylporphyrin, DyLight 680, AAA, DyLight 649, DyLight 633, Magnesium Phthalocyanine, Magnesium Phthalocyanine, Magnesium Tetraphenylporphyrin Merocyanine 540, Naphthalene, Nile Blue (EtOH), Nile Blue, Nile Red, Octaethylporphyrin, Oxazine 1, Oxazine 170, Perylene, Phenol, Phenylalanine, Phthalocyanine, Pinacyanol-Iodide, Piroxicam, POPOP, Porphin, Lucifer Yellow CH, P-Quaterphenyl, Proflavin, P-Terphenyl, Pyrene, Quinine Sulfate, Rhodamine 123, Ethyl-p-Dimethylaminobenzoate, 1,6-Diphenylhexatriene, 2-Methylbenzoxazole, Rhodamine 6G, Rhodamine B, Riboflavin, Rose Bengal, Squarylium dye III, Stains All, Stilbene, Sulforhodamine 101, Tetrakis(o-Aminophenyl)Porphyrin, Tetramesitylporphyrin, Tetraphenylporphyrin, Tetraphenylporphyrin, Tetra-t-Butylazaporphine, Tetra-t-Butylnaphthalocyanine, Toluene, Tris(2,2-Bipyridyl)Ruthenium(II) chloride, Tryptophan.

In one embodiment, the cells are labeled with a detectable label in the presence of a transfection agent. Transfection agents are known and typically are used as carriers for introducing DNA into a cell. The transfection agent may have sufficient molecular size so that it includes a plurality of binding sites for the cell membrane. Although the molecular size for specific transfection agents will vary, most transfection agents can have a molecular weight of at least about 1 kDa, particularly at least about 5 kDa, and more particularly at least about 10 kDa. Illustrative transfection agents include cationic polyaminoacids (e.g., polyallylalanines, poly-L-alanines, poly-L-arginines, poly-L-lysines, and copolymers thereof), spermidines, salmon sperm DNA, poly-L-ornithines, diethylaminoethyl-dextrans, cationic liposomes or lipids, non-liposomal lipids, dendrimers, polynucleotides, and mixtures thereof. Examples of dendrimer transfection agents include those dendrimers having a relatively high electrostatic charge due to (activated) amino and/or carboxyl terminal groups on the outside perimeter of the dendrimer molecule. Such dendrimers can be activated, for instance, by heating up to about 60° C. to selectively remove a portion of the peripheral tertiary amine terminal groups. PolyFect transfection reagent and SuperFect transfection reagent are examples of commercially available activated dendrimers (available from Qiagen GmbH, Hilden, Germany). A commercially available example of a cationic liposome formulation is LipofectAMINE PLUS reagent from Life Technologies, Inc. A commercially available example of a non-liposomal lipid is Effectene transfection reagent from Qiagen GmbH, Hilden, Germany. According to particular embodiments, the transfection agent is a non-viral transfection agent.

Thus, according to one embodiment, the transfection agent does not chemically bond to, or modify, the coating material on the surface of the metal particles.

According to a particular embodiment, the disclosed compositions do not include any therapeutic, diagnostic or bioactive agents other than the detectable label, e.g., a multimode-detectable label. Alternatively, bioactive agents such as nucleic acids (e.g., DNA), or proteins (e.g., antibodies) are conjugated to or associated with the label. The inclusion of such bioactive agents could provide targeting of a label to a specific cell or tissue.

Another option is to label cells in the host in situ so as to allow labeling of structures within the host. This would allow monitoring of labeled structures and cells. In one embodiment, the magnetic particle is targeted to a particular cell expressing a particular marker using an antibody, or fragment thereof.

The living cells for labeling and detection in accordance with the disclosure are those that are of therapeutic, diagnostic, or experimental value when introduced into a patient or host.

### Methods of Imaging

The instant invention provides methods for imaging cells using one or more imaging modalities. In some embodiments the cells are labeled with multiple imaging agents, and in other aspects the cells are labeled with a single labeling agent. In certain embodiments, the single labeling agent is a multimode-detectable agent. The invention provides methods using, for example, the following imaging modalities.

Radionuclide imaging modalities (positron emission tomography, (PET); single photon emission computed tomography (SPECT)) are diagnostic cross-sectional imaging techniques that map the location and concentration of radionuclide-labeled radiotracers. PET and SPECT can be used to localize and characterize a radionuclide by measuring metabolic activity.

PET and SPECT provide information pertaining to information at the cellular level, such as cellular viability. In PET, a patient ingests or is injected with a slightly radioactive substance that emits positrons, which can be monitored as the substance moves through the body. In one common application, for instance, patients are given glucose with positron emitters attached, and their brains are monitored as they perform various tasks. Since the brain uses glucose as it works, a PET image shows where brain activity is high. In certain embodiments of the invention, a cell is labeled ex vivo for PET or SPECT imaging in vivo.

Closely related to PET is single-photon emission computed tomography, or SPECT. The major difference between the two is that instead of a positron-emitting substance, SPECT uses a radioactive tracer that emits low-energy photons.

PET radiopharmaceuticals for imaging are commonly labeled with positron-emitters such as ¹¹C, ¹³N, ¹⁵O, ¹⁸F, ⁸²Rb, ⁶²Cu and ⁶⁸Ga. SPECT radiopharmaceuticals are commonly labeled with positron emitters such as ⁹⁹ mTc, ²⁰¹Tl and ⁶⁷Ga.

Computerized tomography (CT) is contemplated as an imaging modality in the context of the present invention. By taking a series of X-rays, sometimes more than a thousand, from various angles and then combining them with a computer, CT made it possible to build up a three-dimensional image of any part of the body. A computer is programmed to display two-dimensional slices from any angle and at any depth.

In CT, intravenous injection of a radiopaque contrast agent such as those described herein can assist in the identification and delineation of soft tissue masses when initial CT scans are not diagnostic.

CT contrast agents include, for example, iodinated or brominated contrast media. Examples of these agents include iothalamate, iohexyl, diatrizoate, iopamidol, ethiodol and iopanoate. Gadolinium agents have also been reported to be of use as a CT contrast agent (see, e.g., Henson et al., 2004). For example, gadopentate agents has been used as a CT contrast agent (discussed in Strunk and Schild, 2004).

Raman spectroscopy uses energy levels of molecules are probed by monitoring the frequency shifts present in scattered light. A typical experiment consists of a monochromatic source (usually a laser) that is directed at a sample. Several phenomena then occur including Raman scattering which is monitored using instrumentation such as a spectrometer and a charge-coupled device (CCD). Similar to an infrared spectrum, a Raman spectrum reveals the molecular composition of materials, including the specific functional groups present in organic and inorganic molecules and specific vibrations in crystals. Raman spectrum analysis is useful because each resonance exhibits a characteristic ‘fingerprint’ spectrum, subject to various selection rules. Peak shape, peak position and the adherence to selection rules can also be used to determine molecular conformation information (crystalline phase, degree of order, strain, grain size, etc.). Unlike infrared spectroscopy, a single Raman spectrometer can be applied to the molecular characterization of organic and inorganic materials simultaneously. Other advantages of Raman over traditional infrared spectroscopy include the ability to analyze aqueous phase materials and the ability to analyze materials with little or no sample preparation. Deterrents to using Raman spectroscopy as opposed to infrared spectroscopy include the relatively weak nature of the Raman phenomenon and interferences due to fluorescence. In the past several years, a number of key technologies have been introduced into wide use that have enabled scientists to largely overcome the problems inherent to Raman spectroscopy. These technologies include high efficiency solid state lasers, efficient laser rejection filters, and silicon charge coupled device (CCD) detectors.

In Raman spectroscopy instruments, a linear CCD array is typically positioned at the exit focal plane of single stage, low f number Raman monochromators for efficient collection of dispersive Raman spectra. The monochromator disperses the Raman shifted light, and the CCD array typically produces a signal which is proportional to the intensity of the Raman signal vs. wavelength.

Magnetic resonance imaging (MRI) is an imaging modality that is newer than CT that uses a high-strength magnet and radio-frequency signals to produce images. The most abundant molecular species in biological tissues is water. It is the quantum mechanical “spin” of the water proton nuclei that ultimately gives rise to the signal in imaging experiments. In MRI, the sample to be imaged is placed in a strong static magnetic field (1-12 Tesla) and the spins are excited with a pulse of radio frequency (RF) radiation to produce a net magnetization in the sample. Various magnetic field gradients and other RF pulses then act on the spins to code spatial information into the recorded signals. By collecting and analyzing these signals, it is possible to compute a three-dimensional image which, like a CT image, is normally displayed in two-dimensional slices.

Contrast agents used in MR imaging differ from those used in other imaging techniques. Their purpose is to aid in distinguishing between tissue components with identical signal characteristics and to shorten the relaxation times (which will produce a stronger signal on T1-weighted spin-echo MR images and a less intense signal on T2-weighted images). Examples of MRI contrast agents include gadolinium chelates, manganese chelates, chromium chelates, and iron particles. In one particular embodiment, the MRI contrast agent is ¹⁹F.

Both CT and MRI provide anatomical information that aid in distinguishing tissue boundaries. Compared to CT, the disadvantages of MRI include lower patient tolerance, contraindications in pacemakers and certain other implanted metallic devices, and artifacts related to multiple causes, not the least of which is motion (Alberico et al., 2004). CT, on the other hand, is fast, well tolerated, and readily available but has lower contrast resolution than MRI and requires iodinated contrast and ionizing radiation (Alberico et al., 2004). A disadvantage of both CT and MRI is that neither imaging modality provides functional information at the cellular level. For example, neither modality provides information regarding cellular viability.

Optical imaging is another imaging modality that has gained widespread acceptance in particular areas of medicine. Examples of optical imaging agents include, for example, fluorescein, a fluorescein derivative, indocyanine green, Oregon green, a derivative of Oregon green derivative, rhodamine green, a derivative of rhodamine green, an eosin, an erythrosin, Texas red, a derivative of Texas red, malachite green, nanogold sulfosuccinimidyl ester, cascade blue, a coumarin derivative, a naphthalene, a pyridyloxazole derivative, cascade yellow dye, dapoxyl dye and the various other fluorescent compounds disclosed herein.

Another biomedical imaging modality that has gained widespread acceptance is ultrasound. Ultrasound imaging has been used noninvasively to provide realtime cross-sectional and even three-dimensional images of soft tissue structures and blood flow information in the body. High-frequency sound waves and a computer to create images of blood vessels, tissues and organs.

The invention also provides multimodal imaging methods. Certain embodiments of the present invention pertain to methods of imaging a subject, or a site within a subject using multiple imaging modalities that involve measuring multiple signals. In certain embodiments, the multiple signals result from a single label on, or in a cell. As set forth above, any imaging modality known to those of ordinary skill in the art can be applied in these embodiments of the present imaging methods.

The imaging modalities are performed at any time during or after administration of the labeled composition, e.g., labeled cell. For example, the imaging studies may be performed during administration of the labeled cell of the present invention, i.e., to aid in guiding the delivery to a specific location, or at any time thereafter.

Additional imaging modalities may be performed concurrently with the first imaging modality, or at any time following the first imaging modality. For example, additional imaging modalities may be performed about 1 sec, about 1 hour, about 1 day, or any longer period of time following completion of the first imaging modality, or at any time in between any of these stated times. In certain embodiments of the present invention, multiple imaging modalities are performed concurrently such that they begin at the same time following administration of the labeled cell or agent. One of ordinary skill in the art would be familiar with performance of the various imaging modalities contemplated by the present invention.

In some embodiments of the present methods of imaging, the same imaging device is used to perform a first imaging modality and a second imaging modality. In other embodiments, different imaging devices are used to perform the different imaging modalities. One of ordinary skill in the art would be familiar with the imaging devices that are available for performance of the imaging modalities described herein.

### Methods of the Invention

The invention provide methods for imaging cells in vivo by imaging a detectable agent associate with or in the cells. In certain embodiments, the cells are labeled ex vivo and injected or transplanted into a subject. In other embodiments, the cells are labeled in vivo.

In one embodiment, cells are isolated from a donor subject and labeled according to the methods of the invention. In one embodiment, the cells are labeled with a imaging agent, e.g., a multimode detectable agent, ex vivo and introduced into a subject. One particular embodiment includes labeling living cells with a detectable agent to render the cells detectable by one or more imaging modalities, e.g., X-ray, US, Raman, or MR. Such labeled cells may be prepared by simple incubation of cells with the labeling agent in cell cultures. For example, a cell of interest can be cultured in a standard media that includes the labeling agent. Alternatively, the label can be injected in to a subject to label cells in situ, e.g., by injecting into blood vessels, ventricles or other brain or body cavities. The labeling agent may be internalized by a cell via endocytosis and/or diffusion.

The labeled cells may be exogenously applied to a host and monitored within the host using, for example, x-ray, CT, Raman, US or MRI. For example, such cells may be injected or otherwise applied to the host.

Another option is to label cells in the host in situ so as to allow labeling of structures within the host or for tracking movement or migration of cells within a host. For example, tumors could be labeled to monitor effectiveness of treatment and follow metastasis. Labels could be specifically targeted to cells expressing a specific caner marker, e.g., HER2 or EGFR. The cells can be labeled in situ for therapeutic, diagnostic, or experimental purposes. Another embodiment encompasses infusing the magnetic probes into such areas as tumors, so that the growth, metastasis, or regression of the tumor can be monitored. Such a procedure could be part of a treatment protocol to monitor disease progress.

In one embodiment the cells can be stem cells. In another aspect the cells are carcinoma cells. The cells can be directly applied to the area to be treated or studied by means of surgery or injection into the circulation or injection into a structure, organ, or body cavity in situ. When cells are integrated ex vivo into a tissue or organ, such tissue or organ can then be surgically applied or transplanted into a host.

A further embodiment involves using x-ray, US or MRI to monitor the movement, disposition and survival of the cells in the host. When cells are used that are immune cells, which react with a component of a disease process in the host, x-ray, US or MRI monitoring can be used diagnostically to locate the cells attached to the disease process in the host.

Immune cells are understood to encompass lymphoid or myeloid hematopoietic cells.

The cells can be applied to the subject to cure or diagnose a disease or to supply cell type that is lacking or deficient in the host. Additionally, the methods of the invention can assist a clinician to accurately transplant cells into a subject.

In one embodiment the cells can be stem cells. Stem cells are cells that retain their ability to divide and to differentiate into specialized mature cells. Preferably the cells are multipotent cells from the nervous which retain their ability to differentiate into mature cells.

In another embodiment, the cells can be islet cells, transplanted into a subject to cure or alleviate the symptoms of type II diabetes. Islet transplants were first attempted in the 1980s. Initial success rates for islet transplantation in humans were disappointing with only 5% of patients receiving transplants achieving partial function. See Sutherland et al., Evolution of kidney, pancreas, and islet transplantation for patients with diabetes at the University of Minnesota, Am. J. Surg. 166: 456-491 (1993). Amid the failures were isolated success stories of individuals achieving prolonged reversal of their diabetes for a 1 to 2 year period, which encouraged researchers to continue this approach to treatment of diabetes. In 2000, islet transplantations were performed successfully on seven patients with diabetes using a suppression regimen that omitted glucocorticoids, now referred to as the Edmonton protocol. See Ridgway et al., Pancreatic islet cell transplantation: progress in the clinical setting, Treat Endocrinol. 2(3):173-189 (2003). Thus, islet transplantation outcomes have improved markedly. See Shapiro et al., Clinical results after islet transplantation, J. Investig. Med. 49(6): 559-562 (2001); Balamurugan et al., Prospective and challenges of islet transplantation for the therapy of autoimmune diabetes, Pancreas 32(3): 231-243 (2006). Yet, regardless of the optimism generated by these results, barriers to the use of islet transplantation as a practical treatment for diabetes still exist, with one of them being the limited number of donor organs considering that most require multiple transplants to achieve insulin independence.

The invention also provides methods for monitoring the location of transplantation or injection of labeled cells. As indicated above, the labeled cells of the invention can by transplanted into a subject to treat a disease or disorder. In this case, the location of transplantation is important to determining if the cells will have the desired biological activity. The instant invention allows for monitioring the location of transplantation using a cell labeled with a detectable agent and monitoring in real time. Some or all of the cells in a population can contain the label to work effectively in these methods. The location can be further confirmed using one or more additional imaging modalities. In some embodiments, the additional imaging modalities monitor the same imaging agent, e.g., the agent is detectable by multiple modalities.

System

The present invention also provides a Raman system for monitoring cells in vivo comprising a catheter having a first end and a second end with an excitation fiber extending therebetween, the excitation fiber suitable to transmit multi-mode radiation from the first end to the second end to irradiate a target region; a multi-mode laser coupled to the first end of the excitation fiber, the laser generates multi-mode radiation for irradiating the target region to produce a Raman spectrum consisting of scattered electromagnetic radiation; a low-resolution dispersion element positioned to receive and separate the scattered radiation into different wavelength components; a detection array, optically aligned with the dispersion element for detecting at least some of the wavelength components of the scattered light; a processor for processing the data from the detector array to monitor a Raman detectable agent.

Kits

Certain embodiments of the present invention are generally concerned with kits for labeling a cell with an imaging agent. In one embodiment, the kit provides a label and instructions for use. In other embodiment, the kit comprises a labeled cell and instructions for use.

In one embodiment, the kit provides a cryopreserved cell that is labeled with a detectable label. Cells can be cryopreserved by methods that are known to one of skill in the art. For example, methods for cryopreserving cells are disclosed in U.S. Pat. Nos. 6,176,089, 6,361,934, U.S. Pat. No. 6,929,948,U.S. Pat. No. 6,951,712, USPN.

In one embodiment, the invention provides a labeled cell, e.g., a β islet cell, a device for transplanting the cell into a subject and instructions for use. In one embodiment, the cell comprises a multimode-detectable label. In another embodiment, the instructions for use pertain to confirming the location of transplantation.

### EXAMPLES

It should be appreciated that the invention should not be construed to be limited to the examples that are now described; rather, the invention should be construed to include any and all applications provided herein and all equivalent variations within the skill of the ordinary artisan.

### Example 1

### Labeling of Stem Cells with Detectable Labels

In a New Zealand white rabbit 5 mL of bone marrow was be drawn into a 20-mL syringe with an 18-gauge needle. After the bone marrow was collected it was mixed with an equal volume of PBS to homogenize thoroughly until all blood clots are dissociated. The cell suspension was then centrifuged for 10 min at 900 g. The supernatant was then aspirated and the pellet was resuspended in PBS to a final density of 4×107 nucleated cells/mL. The cell suspension was then layer over a 1.073 g/mL Percol solution. This preparation was then centrifuged at 900 g for 30 min. The middle phase of the resulting three phases was collected and centrifuged again for 10 min at 1000 rpm. The supernatant was then removed and the pellet was resuspended in 1 mL of PBS. This preparation was then centrifuged again and the supernatant was removed. Bone-marrow-derived rabbit mesenchymal stem cells were cultured in Dulbecco's modified Eagle's medium (DMEM), 10% FBS, 100 units/mL penicillin and 100 μg/mL streptomycin and 10 μg/mL insulin.

For electroporation MSCs as cultured in example 1 were first trypsinized to free from culture flasks and washed two times in PBS. After wash, cells were suspended in phosphate-buffered saline (PBS) at a density of 1-5×106 cells/mL in sterile 0.4-mm-gap electroporation cuvettes. Dex-Gold 50 (Nanocs) was added at 250-2000 μg Fe/mL. Cells were electroporated using a BTX electroporation system under a variety of conditions at 100 V for 15 ms. After electroporation treatment, cells were left in the cuvette holder for 1 min, transferred, and left on ice for 5 min. A small top layer of foam was removed and cells were washed twice for further use. Labeled cells had a pinkish hue.

For poly-1-lysine assisted labeling MSCs as described herein were used. To this end, 30 μg/ml Gold-dextran particles (Nanocs) was mixed with Protamine sulfate (300 ng/mL. American Pharmaceuticals Partner Bedford, Ohio) incubated for 1 hour, and added to the cell culture medium as described in example 124 hours.

For protamine assisted labeling MSCs as cultured in example 1 were used. To this end, 30 μg/ml Gold-dextran particles (Nanocs) was mixed with poly-L-lysine (375 ng/ml, Sigma-Aldrich, St. Louis, Mo., USA), incubated for 1 hour, and added to the cell culture medium as described in example 1 for 24 hours.

Synthesis of gold or silver tat particles. 50 nm gold or silver detran coated particles (Nanocs) were first stabilized by crosslinking the dextran coating with epichlorohydrin and then reacted with ammonia to yield amino groups on the dextran coating for further modification. The particles were then reacted with N-succinimidyl-3-(2-pyridyldithio) propionate (SPDP) in pH 7.4, sodium acetate buffer for 3 h, and unreacted SPDP was removed by gel filtration (Sephadex G-25; Sigma, St. Louis, Mo.). The commercially available TAT peptide, FITC-LC-TAT from Anaspec (Jose, Calif.) was added to the purified solution, and the reaction mixture was stirred at room temperature for 3 h. The final product was separated from by-products using a G-25 gel filtration column. For TAT-peptide assisted labeling MSCs as cultured in example 1 were used. To this end, 30 μg/ml of the Gold-TAT peptide prepared as described above was added to the cell culture medium as described in example 1 for 24 hours.

Cells labeled as described above were fixed for 10 min with PBS containing 4% paraformaldehyde followed by washing with PBS. For immunohistochemistry the following primary antibodies were used for anti dextran staining, mouse monoclonal No. 14533 (StemCell Technologies, Vancouver, Canada, 1:1000) and for secondary antibody goat anti-mouse 488 (1:300) from Molecular Probes, Eugene, Oreg., USA was used. Tissue sections or culture dishes were incubated overnight at 4° C. with primary antibodies diluted in 0.1 M PBS containing 10% normal goat serum and then with the appropriate secondary antibodies for 2 h at room temperatures. Negative controls were prepared identically, except for the omission of primary antibody. Cells were embedded with Vectashield mounting medium containing DAPI as nuclear counterstain (Vector, Burlingame, Calif., USA). Immunofluorescence analysis was performed using Olympus BX51 and IX71 epifluorescence microscopes equipped with an Olympus DP-70 digital acquisition system. Imaging revealed high labeling efficiency for all techniques described herein.

### Insertion of SH Groups in Aminodextran

5 g of aminodextran with a molecular weight of approximately 40,000 and a concentration of approximately 30 amino groups per molecule were dissolved in 200 ml of phosphate buffer, pH 8.5. 1.225 g SATP were dissolved in 62 ml DMSO and added to the aminodextran solution. The mixture was stirred at room temperature for 3 hours and then dialyzed. The release of the SH groups was achieved by adding 694 mg of hydroxylamine dissolved in 2 ml water. The pH was adjusted to 6.0 and after one hour dialyzed against phosphate buffer, pH 6.0. Uncoated gold nanoparticles or silver nanoparticles (Nanocs) were adjusted to a pH of 5.6. Subsequently, 400 □g of the SH aminodextran produced was added to 10 mM K acetate, pH 5.6, and stirred for 2 hours.

Insertion of Maleimide Groups (MH⁺) into the Antibody18 mg immunoglobulin of a monoclonal murine antibody against digoxigenin (MAB<Dig>M-IgG) were dissolved in 1 ml phosphate buffer, pH 7. 6.9 mg of maleinimidohexyl-N-hydroxysuccinimide (MHS) were dissolved in 690 μl DMSO. 330 μl of the antibody solution were subsequently mixed with 10 μl of the MHS solution and stirred at 4 C for 2 hours. Afterwards dialysis was performed overnight.

Production of the Conjugates from MH-IgG and SH-Aminodextran Gold 3600D of the SH-aminodextran gold were adjusted to pH 6.6. Subsequently 720 μg of the MH-modified antibody, dissolved in 40 ml acetate/TRIS buffer pH 6.6 were added, the pH adjusted to 7.0, and the mixture stirred at 4 C overnight. The surplus SH and maleimide groups which had not completely reacted were then stopped by adding thioglucose and iodo-acetamide. Subsequently, 400 mg of bovine serum albumin were added and the pH adjusted to 7.8.

### Production of an Adsorptively Loaded Conjugate

The solution of the antibody to be loaded was dialysed against the appropriate loading buffer, TRIS, pH 8.0. Possibly existing aggregates were removed by filtration. The pH value of the gold sol solution was adjusted to the pH of the protein solution. The antibody solution (10 μg protein/OD gold) was added to the gold sol solution and incubated for 2 hours. Subsequently it was saturated by adding a 10% BSA solution (final concentration approximately 1% BSA). Purification of the conjugate was reached by dialysis.

Emulsions suitable for use in cell labeling may be prepared, for example, by adding two parts by volume of a brominated perfluorocarbon to 1 part by volume of lactated Ringer's solution containing a small amount (e.g., 6%) of an emulsifing agent, e.g., Pluronic F-68, and agitating on a vortex or sonicator until a stable emulsion is formed. More concentrated emulsions are formed by adding neat perfluorocarbon, up to a ratio of 12:1 by volume, and mixing until a stable emulsion is formed.

Concentrated emulsions of this type, particularly those having perfluorocarbon/aqueous phase ratios of 6:1 to 10:1, are useful in medical applications of low number of cells therefore requiring a high degree of radiopacity. While the toxicity of the compounds of the invention appears to be greater than that of monobrominated acyclic fluorocarbons, the greater radiopacity permits smaller amounts of radiopaque to be used, thus overcoming the toxic effects.

### Phantom Creation

For phantom creation gold-dextran labeled MSCs were suspended in 4% gelatin. The appropriate number of cells were injected directly into a gelatin bed to create approximate point sources.

### Ultrasound Imaging

Sonography was performed with a L25E 13-6 MhZ probe on a Micromaxx US system (Sonsite). Grayscale imaging was performed with a center probe frequency of 6.00 MHz, a dynamic range of 55 dB, and a persistence setting of two.

### Example 2

### Labeling and Transplantation of Islet Cells

The following example describes the labeling and transplantation of β islet cells and the imaging of these cells when transplanted in to a subject.

Fresh human cadaveric islets were provided by the Joslin Diabetes Research Center (Boston, Mass.) under an approved protocol of the National Islet Cell Resource Program. Islets were cultured in RPMI 1640 medium (Gibco), supplemented with 10% fetal calf serum and 1% penicillin/streptomycin/L-glutamine (all reagents from Sigma Co) in a humidified CO2 incubator at 37° C. and a 5% CO2 atmosphere. Islets were cultured in tissue culture plates and culture media was replaced every 3 days. For all Feridex labeled islets, islets were initially labeled with procedure described below and then were cultured in contrast free medium. In the case of PFC labeled islets, the respective PFC was supplemented to each change of culture medium throughout the entire culture period.

Labeling of Islets with Feridex

Human islets were labeled with the commercially available SPIO, Feridex (Berlex Laboratories). Islets were incubated with 25 μg/ml of Feridex in culture medium overnight at 37° C. Islets were then thoroughly washed with PBS to remove all extracellular contrast agent.

Labeling of Islets with Perfluorocarbon Emulsions

The PFC agents were composed of perfluoro-15-crown-5 ether (Exfluor Research) or perfluorooctylbromide (Sigma Co.) that was emulsified (40% vol/vol) in a mixture of H2O and 5% lecithin which yielded a particle size of approx100-200 nm. Specifically emulsions were prepared by The critical aspects observed so far are to sonicating at 40% power the lecithin-water mixture (5% lecithin in water w/v) until the solution is almost transparent. Next, the respective PFC was added to the lecithin-water mixture (40% PFC v/v) and sonicated until a milky homogenous suspension was formed. For labeling of cells 4 μl of this emulsion was added for each ml of culture media. Culture media enriched with PFC emulsions was then sonicated at 40% power. The resulting solution was then filtered through a 0.22 μm filter. The mix was then added to islet cells and incubated for at 37° C. in 5% CO2. When removed from culture cells were washed three times with PBS to remove excess PFC. Coupling PFCs with rhodamine allowed for detection of intracellular PFCS using fluorescence microscopy.

Viability of labeled human islets was determined using a microfluorometric assay. Cells were incubated for 30 minutes with 10 mM Newport Green (NG, Sigma, St. Louis, Mo.), which stains viable cell cytoplasm green. Following NG staining, islets were incubated for 10 minutes with 5 mM propidium iodide (PI, Sigma, St. Louis, Mo.), which emits red fluorescence when bound to nucleic acids. NG was excited by using a 500 nm laser line and emitted fluorescence was detected using a 535 nm long-pass filter. PI was excited by using a 514 nm laser line and emitted fluorescence was detected using a 550 nm long-pass filter. NG stained cells (green) were counted as viable, while PI stained cells (red) were counted as dead. In cases of dual staining, the cells were considered dead.

### MTS Assay

The metabolic assimilation rate (indicator of cellular toxicity) of islets cells in response to increasing concentrations of Feridex, PFPE, and PFOB was determined using an MTS (3-(4,5-dimethylthiazol-2-yl)-5-(3-carboxymethoxyphenyl)-2-(4-sulfophenyl)-2H-tetrazolium) assay (CellTiter 96 AQueous one solution cell proliferation assay, Promega). MTS is a tetrazolium salt that is cleaved to form a formazan dye only by metabolically active cells. After overnight incubation, 100 L of MTS was added to each well. Plates were incubated at 37° C. for 1 h followed by measurement of the absorbance at 492 nm using a microplate reader (Beckman Coulter). The absorbance values for the different labeling conditions were calculated as a percentage of the absorbance for unlabeled control cells. This procedure was repeated at 18 and 24 hours after labeling.

### Detection of Intracellular Feridex

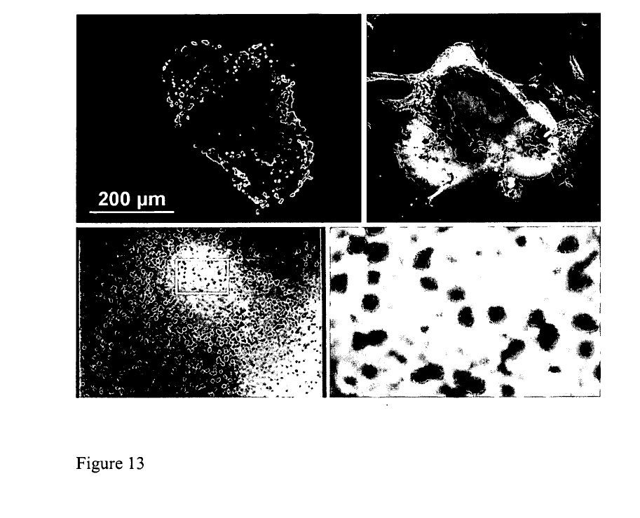

In order to detect the ferric iron in the MCs, sections were stained with Perl's reagent in order to precipitate a Prussian Blue product. To this end, islets were firmly affixed to a glass slide using a cytospin. Islets were then fixed with glutaraldehyde and incubated for 30 min with 2% potassium ferrocyanide (Perls' reagent) in 6% HCl, washed, and counterstained with nuclear fast red. In addition, we used immunofluorescent staining in order to detect dextran (the polymer in the Feridex coat) which, in our experience, appears to be more sensitive than Prussian Blue staining. See FIGS. 13A-D. Briefly, samples were incubated overnight at 4° C. with mouse anti-dextran IgG primary:antibody (1:100 diluted, Stem Cell Technologies) in 0.1 M PBS containing 10% normal goat serum. After washing, goat anti-mouse-594 secondary antibody (Molecular Probes) was added for 2 h at room temperature. Immunofluorescence analysis was performed using Olympus BX51 and IX71 epifluorescence microscopes equipped with an Olympus DP-70 digital acquisition system.

Immunostaining of MCs was performed using an anti-dextran antibody to visualize the presence of dextran-coated Feridex particles within MCs, as described previously for direct Feridex labeling of cells (Walczak, Kedziorek et al. 2005). Briefly, samples were incubated overnight at 4° C. with mouse anti-dextran IgG primary antibody (1:100 diluted, Stem Cell Technologies) in 0.1 M PBS containing 10% normal goat serum. After washing, goat anti-mouse-594 secondary antibody (Molecular Probes) was added for 2 h at room temperature.

### Insulin Secretion Assay

A static incubation assay was used to assess the insulin secretion response of labeled human islets. One hundred islets were placed in a culture insert (membrane pore diameter 12 μm; Millicell PCF) in six-well plates. The insulin secretion was measured after 1.5 hrs in a solution of a specific glucose level. Specifically, a step-wise increase in glucose concentration from 6 mM to 8 mM D-glucose in RPMI 1640 medium was used to assess the fine glucose responsiveness of encapsulated cells. Aliquots of the medium were stored at −80° C. The C-peptide content of the samples was determined with an enzyme-linked, immunosorbent assay (ultrasensitive human c-peptide ELISA, Alpco Diagnostics); results (in ng/ml) were expressed as the means of three independent experiments. The C-peptide secretion was also assessed at 7 days and at 14 days following islet encapsulation, using 8 mM glucose and 90 min incubation.

### Renal Subcapsular Transplantation of Islets in Mice

PFOB-labeled islets were grafted beneath the renal capsule of the left kidney of recipient C57B1 mice. The animals were anesthetized with an intra-peritoneal injection of a mixture of ketamin (50 mg/kg) and acepromazin (5 mg/kg). The right kidney was exposed through an abdominal incision and encapsulated islet cells were implanted under the renal capsule. The incision was sutured and the animals were then allowed to recover or were sacrificed for ex-vivo imaging. A total of 2,000 islets were transplanted. The right kidney was used as a control.

### Fluoroscopic MR Imaging

MR imaging was performed using a 9.4T Bruker BioSpin MRI GmbH equipped with an additional preamplifier for F-19 spectroscopy. For radio-frequency transmission and detection at F-19 frequency (59.87 Mhz), a linearly polarized resonator was used. A standard T-2 weighted spin echo (SE) pulse sequence was employed. SE parameters were TR/TE=1500/15 ms; FOV 3×3 cm; matrix 128×64; slice thickness 1 mm; NAV=1. The pulse sequence was repeated continually for a total time of 1 minute 4 seconds. Segmentation and 3d reconstruction was done using the imaging software Amira.

### CT Imaging

Images were obtained using a Gamma Medica XSPECT scanner. CT subjects were placed on an animal bed and anesthetized with 0.25% isoflurane flowing at 0.5 L/min throughout the imaging with exposure to radiation limited to a maximum of 30 minutes. For each scan, 1024 projections with 1024×1024 pixels were obtained at different angles of view between 0° and 360°. Acquisition time for each view was 1 second. Scanning was performed in a clockwise direction with an X-ray tube to detector distance of 269 mm and an X-ray tube to COR distance of 225 mm. Images were obtained in rotation steps of 0.703° with respective voltage and current of 50 kVp and 600 μA. Segmentation and 3d reconstruction was done using the imaging software Amira.

### Statistical Analysis

Statistical analysis was conducted using a Students T-test with a significance level P<0.05. Data were also analyzed using the bioequivalence (BE) test. The test was performed using the Two-One Sided T-test approach (TOST) (Jacobson and Poland 2005). In a BE test, the null hypothesis is that two groups differ by an amount 0 or more. In TOST, the null hypothesis is rejected and two groups are declared bioequivalent at the type I error rate θ if a (1-2θ) confidence interval is contained in (−θ, θ). Because no θ value has been established for declaring bioequivalence in islet cell viability, we report the lowest value that would allow the two samples to be declared bioequivalent, with θ being reported as a percent difference from control. All statistical analysis was done using the statistical software R.

### Results

### Islet Viability

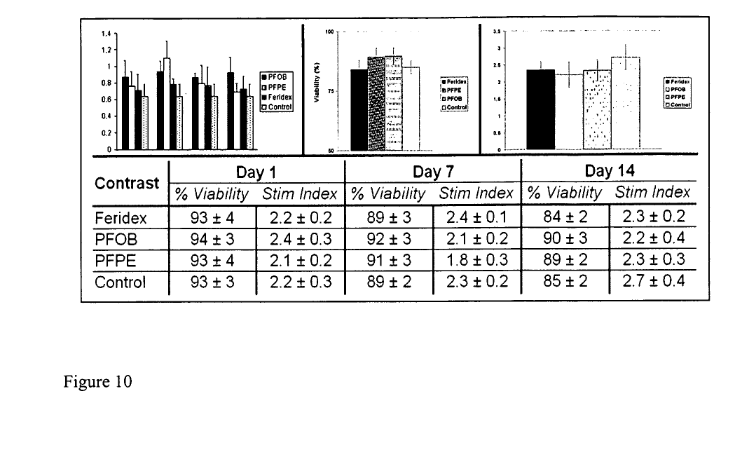

Differences in viability between human islets labeled with Feridex, PFPE, and PFOB compared to unlabeled islets were assessed at days 1, 7, and 14. In general, the percentage survival between Feridex labeled islets and control islets showed no significant statistical difference over all days (p>0.05). See FIGS. 10A-D. However, percentage survival between PFPE labeled islets and control islets showed significant statistical differences (p<0.05) on days 7 and 14. A statistically significant increase in percentage viability over controls was found with PFOB labeled islets at all time points including 24 hours after labeling.

### Cell Proliferation

Cell proliferation was measured using an MTS assay. In general Feridex and PFC labeled islets showed an increase in cell proliferation when compared to unlabeled islets for all label concentrations.

### Glucose Responsiveness

Insulin secretory response of labeled islets was compared against unlabeled islets. To detect any specific difference in insulin secretion, islets were incubated in solutions of 6 mM and 8 mM glucose. The glucose responsiveness stimulation index, defined as the increase of insulin secretion after changing from 3 mM glucose to 6 mM glucose, was found to be 2.19, 2.07, and 2.40 for PFOB labeled islets, PFPE labeled islets, and unlabeled islets, respectively. Additionally, there was no significant statistical difference (p>0.05) in glucose responsiveness stimulation index between PFOB and PFPE which was confirmed with a BE test θ value <10%.

### NMR of PFCs

PFOB must be present in solution in a micellar form because the chemical shifts in solution are virtually identical to those seen for neat PFOB and because the aqueous solubility of PFOB is below the limit of detection for the present ¹⁹F NMR experiments. In the ¹⁹F NMR spectrum of PFOB, eight resonance peaks are observable, one for each carbon position.

### Imaging of Islets

Labeling of islets with PFCs allowed non-invasive tracking of transplanted islets in mice. Coupling PFCs with rhodamine and then imaging under fluorescence microscopy revealed that the PFC label is incorporated into approximately 80% of the islet. Using CT imaging, PFOB labeled islets were identified in vivo after transplantation into the kidney of mice. While individual islets could not be resolved, small groups of islets were clearly identifiable. Under high resolution ¹⁹F MRI, PFOB labeled islets were clearly distinguishable from soft tissue after transplantation into the kidney of mice.

### Discussion

Using magnetic resonance (MR) imaging, it is possible to track the delivery and biodistribution of cells when these cells are magnetically pre-labeled (Kraitchman, Tatsumi et al. 2005). In diabetes research, magnetic labeling of islet cells has been applied in rodents for MR monitoring of islet grafting (Koblas, Girman et al. 2005; Evgenov, Medarova et al. 2006). This has allowed a precise determination of islets after transplantation under the kidney capsule of rodents. Moreover, it has allowed assessment of graft rejection in syngeneic grafts vs allografts (Kriz, Jirak et al. 2005). Only in the allogeneic group did the number of hypointense spots gradually decrease until approximately 35% of the initial count remained suggesting destruction of the allogeneic, but not the syngeneic cells (Kriz, Jirak et al. 2005).

Recently, MRI cell tracking using Feridex/Endorem has been introduced into the clinic (de Vries, Lesterhuis et al. 2005; de Vries, Lesterhuis et al. 2005). This Phase I clinical trial has proven that MRI cell tracking utilizing SPIOs is a clinically safe and feasible procedure. In this patient study, Endorem and ¹¹¹Indium oxine-labeled dendritic cells, primed with melanoma antigens, were used as cancer vaccines to boost the immune system of melanoma patients. To this end, cells were injected in draining lymph nodes under ultrasound guidance. While the main aim of the study was to determine cell migration to nearby lymph nodes, a surprising finding was that cells appeared to be misinjected in half the patients. Only with MRI, and not radionuclide imaging, could it be determined that cells were accidentally misinjected into either the surrounding muscles or perinodal fat rather than the target lymph node. These results demonstrate the importance of MR-labeled cells, not only in assessing cell biodistribution and migration following injection, but also to guide, using MR-compatible devices, correct targeting of the initial injections.

In addition to demonstrating the strength of MR cell-tracking, this study also highlights one of the main obstacles of MR tracking of cells in patients. In this particular study a relatively large number of cells were injected at a single point source. In this case, the required delivery strategy for therapeutic efficacy also maximized MR detectability as a large payload of contrast agent was localized to a single area of known origin. For many other cellular therapeutic applications, such as those that employ intravascular delivery of cells, the broad distribution of cells poses a major obstacle. As compared to direct point source injection, intravascular delivery will result in cells distributed throughout the body at much lower local density. As many areas throughout the body appear hypointense on T2* weighted MR, localization of SPIO labeled cells after intravascular delivery is particularly problematic.

Due to the inherent difficulty of detecting cells that are broadly distributed, the majority of studies, including this one, examining the in vivo detection of pancreatic islets rely on a point source injection in the kidney capsule instead of intraportal infusion as called for by the Edmonton protocol. Tai et al. demonstrated that at 1.5T a cluster of 200 islets was necessary for in vivo detection (Tai, Foster et al. 2006). In terms of clinical translatability and applicability to detection of islets with the Edmonton protocol, it is highly unlikely that intraportal infusion will result in 200 islet clusters distributed throughout the liver. For this reason, a sensitive tracking of SPIO labeled islets after intraportal administration appears to be difficult at best.

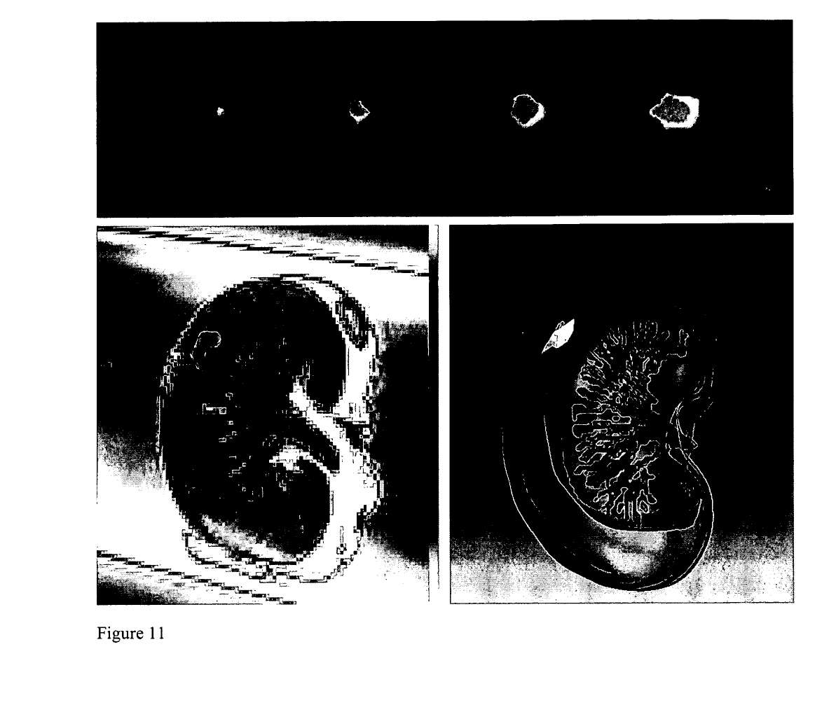

As opposed to SPIOs that create signal voids on T2* weighted MR scans, perfluorcarbons can be used in conjunction with ¹⁹F MRI to create positive signal. Fluorinated contrast agents take a different approach to molecular labeling. Fluorinated contrast agents are detected directly by ¹⁹F MRI, assuring a lack of uncertainty about the signal source as the body lacks any endogenous fluorine. The fluorine signal also offers a hotspot interpretation when superimposed on anatomical ¹H MRI scans, which can be taken during the same session (FIG. 11A-C). By overcoming the limitations associated with traditional ¹H MRI contrast agents, fluorinated agents are able to effectively and accurately track transplanted cells. PFCs, PFOB and PFPE are advantageous as contrast agents because both compounds are visible under ¹⁹F MRI (Caruthers, Neubauer et al. 2006; Cyrus, Abendschein et al. 2006), which offers a greater range of sensitivity to the local environment than ¹H MRI because of fluorine's 7 outer-shell electrons. The perfluorocarbon perfluoropolyether (PFPE) has been used label dendritic cells and has been shown to have no effect on dendritic cell proliferation, function, or maturation (Ahrens, Flores et al. 2005). Additionally, PFPE is an ideal ¹⁹FMR contrast agent as all fluorine atoms are biologically equivalent giving a single peak on MR as compared to the multiple peaks produced by PFOB. Both PFCs are attractive in terms of theoretical safety as they are thought to be biologically inert and therefore cannot be broken down unlike most metal-based contrast agents.

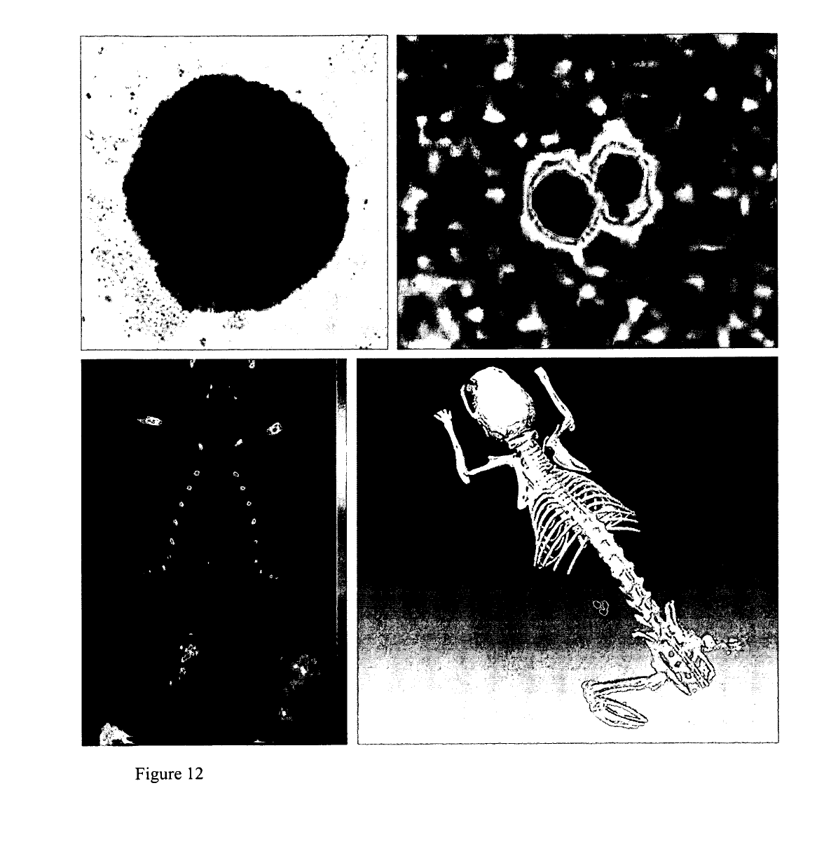

In addition to ¹⁹F MRI, PFOB labeled islets proved to be detectable with CT. In perfluoroctylbromide all the hydrogen atoms are replaced by 17 fluorine atoms and 1 bromine atom. This agent is useful as both a radiographic and an MR contrast agent. The attached bromine results in its radiopaque characteristics, and the lack of hydrogen atoms results in a lack of signal generation with MRI. Since it does not generate signal intensity, perflubron appears as a negative contrast agent. Further the fluorine present in PFOB allow for its detection with ¹⁹F MRI. This allows for labeled cells to be distinguished from bone and other dense tissues, as demonstrated by in vivo imaging results in mice (FIG. 12A-D). As a result, after transplantation, CT can be used to locate small clusters of cells with respect to gross skeletal anatomy. Currently the highest resolution of micro-CT is approximately 5 microns which could potentially enable single cellular detection. This resolution far surpasses current high-resolution clinical scanners, with minimal slice thickness of approximately 1 mm (Robinson 2004). For this reason, the clinical translatability of cell tracking with CT is limited. Nevertheless, for particular applications in which visualization of a point injection of a large numbers of cells or imaging of cell clusters such as pancreatic islets is desired, CT imaging may prove useful.

PFOB is also visible under ultrasound (Schutt, Klein et al. 2003), currently marketed as the ultrasound contrast agent Oxygent®. By having detection under three imaging modalities using a single contrast agent, labeled cells could be tracked from the moment of transplantation to the final migration site. PFOB labeled islets could be accurately transplanted using ultrasound guided injection. Using CT, the islet transplantation site could be located with respect to skeletal anatomy. Upon detection of the relative transplantation site, ¹⁹F MRI in conjunction with ¹H MRI could be used to confirm the transplantation site and offer a distinction from soft tissue with its high spatial resolution. This combination of ultrasound, CT, and ¹⁹F MRI visibility makes PFOB an ideal contrast agent for in vivo cell tracking.

A secondary aim of this study was to assess whether the use of PFCs could also be beneficial for the secretory activity and overall viability of cultured purified islets before transplantation. Studies have shown that labeling pancreatic islet cells with superparamagnetic iron oxide contrast agents led to a significant decrease in insulin secretion, compared to unlabeled cells (Kriz, Jirak et al. 2005). PFOB overcomes these limitations of cellular function and viability. Our insulin secretion assays have shown that there is no significant difference between the glucose responsiveness of PFOB labeled and unlabeled islets demonstrating that PFOB has no significant change in islet function. Additionally, islet viability data shows that there is a significant increase in viability in PFOB labeled islets compared to both Feridex labeled and unlabeled islets. This is most likely due to PFOB's ability to attract oxygen molecules facilitating an increase in gas exchange. PFOB is marketed both as LiquiVente (Allianc Pharmaceuticals) (Wakabayashi, Tamura et al. 2006), an oxygen carrying liquid drug, and Oxygent® (Alliance Pharmaceuticals), a blood substitution agent, both currently undergoing phase 3 clinical trials. PFCs have been successful blood substitution agents in clinical trials. Perfluorocarbons (PFCs) have a high oxygen solubility coefficient and maintain high oxygen partial pressures for extended time. They serve also as oxygen “reservoirs” for harvested organs in pancreas organ transplantation (Ricordi, Fraker et al. 2003; Ramachandran, Desai et al. 2006) (Brandhorst, Iken et al. 2005) (Bergert, Knoch et al. 2005; Takahashi, Tanioka et al. 2006).

By directly labeling islets with CT and ¹⁹F MRI visible PFOB, we have developed a way to effectively track cells in vivo after transplantation. PFOB, marketed as an oxygen carrying liquid drug, increases the viability of labeled islets making it a suitable alternative to traditional ¹H MRI contrast agents such as lanthanide complexes and heavy metals which have displayed toxicity issues in previous studies. Moreover, the lack of endogenous fluorine ensures the authenticity of the signal when imaging with ¹⁹F MRI. In addition, overlaying ¹⁹F MRI scans on anatomical ¹H MRI scans taken during the same session allows for ‘hot-spot imaging’ and accurately confirms the location of transplanted islets even when in soft tissue. This study represents the first attempt at cell labeling with a radioopaque contrast agent for detection with x-ray modalities. Bromofluorocarbon also represent the first reported trimodal contrast agent for cell tracking. Visibility of labeled cells with CT overcomes the small field of view limitations associated with MRI and also allows groups of labeled islets to be distinguishable from bone. In clinical applications, transplanted islets could be distinguished from skeletal anatomy using CT followed by confirmation using ¹⁹F MRI. As superimposition of CT and MRI scans, using hybrid X-Ray/MR imaging systems (Fahrig, Heit et al. 2003; Ganguly, Wen et al. 2005), become more prevalent in the future, multimodal contrast agents, such as PFOB, will allow researchers and clinicians to accurately monitor labeled cells in vivo.

### Example 3

### Gold Labeled Mesenchymal Stem Cells For Trimodal Detection on Raman Spectroscopy, Ultrasound and X-ray Modalities

Currently the majority of image-guided interventional procedures are performed with x-ray fluorscopy and ultrasound (US). In order to track cellular delivery and engraftment with commonly used modalities we have developed a novel gold-dextran nanoparticle labeling technique that enables x-ray and ultrasound (US) visualization of mesenchymal stem cell (MSCs). To ensure the specificity of signal on x-ray and US, we further explored the use of Raman spectroscopy to detect a unique spectral signature of gold-dextran particles.

### Materials and Methods

MSCs derived from the bone marrow of adult New Zealand White rabbits were isolated as previously described and expanded in culture. Cells were labeled with gold-dextran mixed with transfection agent poly-L-lysine or protamine sulfate added at 30 μg Au/ml to the cell cultures for a 24-hour incubation. Viability and proliferation rates of labeled cells were determined by trypan blue dye exclusion and MTS assay. Gold-dextran uptake was visualized by anti-dextran immunohistochemistry. Non-invasive imaging was used to assess detection sensitivity in agarose phantoms and monitor cell delivery after rabbit hind-limb injection. For Raman spectroscopy a 100 mW 532 nm green diode laser was focused through a lens with 10 cm long focal length. The Raman scattered light was collected by a 600 micron core multi-mode fiber and delivered to a Si—CCD based spectrometer to monitor the scattered light spectrum.

### Results

MSCs were readily labeled with gold-dextran particles as determined by immunohistochemistry. Post label viability was 95±6.1% at day 1 and remained at 93±4.2% after 1 week following labeling. MTS assay showed no-statistically significant difference from unlabelled cells. Gold labeled MSCs were readily detected on a 64-slice CT clinical scanner at a minimum concentration of ten thousand cells and by clinical grade US at a concentration of one hundred thousand cells. A point source injection of one million gold labeled MSCs were visible immediately and at 2 weeks post-injection in rabbit hindlimb with both x-ray fluoroscopy and CT. Gold-dextran particles revealed a strong fluorescent enhancement at 3350 cm⁻¹ which corresponds to O—H stretch and a fluorescence enhancement at 6400 cm⁻¹.

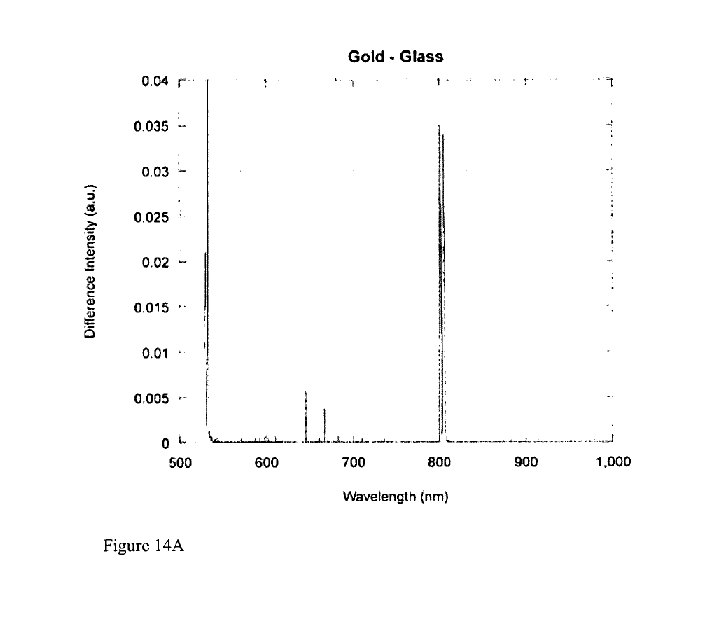

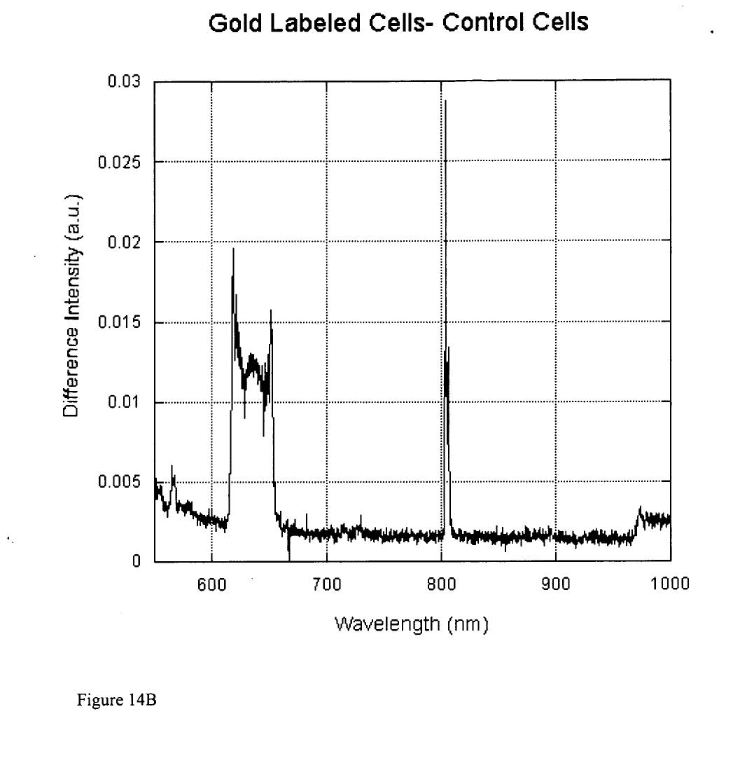

Compared to a reference container which only contains water, a large Raman signal enhancement (˜four times) was observed at 3350 cm⁻¹ which corresponds to O—H stretch. In addition we observed a strong fluorescence enhancement at 6400 cm⁻¹. See FIGS. 14 and 15.

### Conclusion

Gold particle labeling offers a new approach for immediate visualization of cell injection success using conventional X-ray fluoroscopy, US, CT and/or Raman Spectroscopy.

### Incorporation by Reference

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of ex-vivo labeling of a cell for in vivo imaging comprising:
contacting a cell ex vivo with a labeling agent such that cell becomes labeled;
thereby labeling a cell for in vivo imaging.

2. The method of claim 1, wherein the cell is transplanted into a subject.

3. The method of claim 3, wherein the labeling agent is detectable by a modality selected from the group consisting of X-ray, CT, ultrasound, Raman, and magnetic resonance.

4. The method of claim 1, wherein the labeling agent is a multimode-detectable labeling agent.

5. The method of claim 4, wherein the labeling agent is detectable by at least two modalities selected from the group consisting of X-ray, CT, ultrasound, Raman, and magnetic resonance.

6. The method of claim 1, wherein the cell is a cell for use in cellular therapy.

7. The method of claim 5, wherein the cell is a immune cell, stem cell, progenitor cell, islet cell or other cell with regenerative properties.

8. The method of claim 7, wherein the labeling agent is a perfluorocarbon (PFC).

9. The method of claim 8, wherein the PFC is perfluoro-15-crown-5-ether (PFCE), perfluorooctylbromide (PFOB).

10. The method of claim 1, wherein the labeling agent is a colloidal metal particle.

11. The method of claim 10, wherein the colloidal metal particle is a colloidal gold particle.

12. The method of claim 9, wherein the particle is a core-shell particle.

13. The method of claim 12, wherein the shell of the core-shell particle is derivatized with functional groups for the conjugation of a bioactive molecule.

14. The method of claim 13, wherein the bioactive molecule is a peptide or polypeptide.

15. The method of claim 14, wherein the peptide or polypeptide is an antibody or fragment thereof.

16. The method of claim 1, wherein the labeling agent is a gold-based agent, a silver-based agent, an iron-based agent, or a gadolinium-based agent.

17. The method of claim 16, wherein the labeling agent is magnetic, paramagnetic or superparamagnetic.

18. The method of claim 1, wherein the cell is contacted with the labeling agent in the presence of a transfection agent.

19. The method of claim 1, wherein the cell is electroporated in the presence of a labeling agent.

20. A method of ex vivo labeling of a pancreatic β islet cell for in vivo imaging, comprising:
contacting the cell with a labeling agent ex vivo;
thereby labeling the cell.

21. The method of claim 20, wherein the labeling agent is a multimode-detectable labeling agent.

22. The method of claim 20, wherein the labeling agent is detectable by a modality selected from the group consisting of X-ray, CT, ultrasound, Raman, and magnetic resonance.

23. The method of claim 21, wherein the labeling agent is detectable by at least two modalities selected from the group consisting of X-ray, CT, ultrasound, Raman, and magnetic resonance.

24. The method of claim 23, wherein the labeling agent is a perfluorocarbon (PFC).

25. The method of claim 24, wherein the labeling agent is radiopaque.

26. The method of claim 25, wherein the PFC is perfluoro-15-crown-5-ether (PFCE), perfluorooctylbromide (PFOB).

27. The method of claim 20, wherein the labeling agent is PFOB.

28. The method of claim 27, wherein the β islet cell is transplanted into the kidney of a subject.

29. The method of claim 28, wherein the labeled cell is imaged by CT and MR imaging.

30. The method of claim 29, further comprising imaging the labeled cell using ultrasound.

31. A method for accurately transplanting cells into a subject comprising;
labeling cells with an imaging agent;
guiding the injection of labeled cells using a first mode of detection;
thereby accurately transplanting the cells.

32. The method of claim 31, wherein the imaging agent is a multimode-detectable imaging agent.

33. The method of claim 32, further comprising confirming the accuracy of injection using a second mode of detection.

34. The method of claim 33, further comprising confirming the accuracy of injection using a third mode of detection.

35. The method of claim 31, wherein the first mode of detection is ultrasound.

36. The method of claim 33, wherein the second mode of detection is MR.

37. The method of claim 34, wherein the third mode of detection is CT.

38. The method of claim 31, wherein the agent is a PFC or a colloidal metal particle.

39. The method of claim 38, wherein the agent is PFOB.

40. The method of claim 31, wherein the cell is a β islet cell is transplanted into a kidney.

41. A method for accurately transplanting cells into a subject comprising;
labeling cells with a multimodal imaging agent;
guiding the injection of labeled cells using ultrasound detection;
thereby accurately transplanting the cells.

42. The method of claim 41, further comprising confirming the accuracy of injection using MR or CT imaging.

43. The method of claim 41, further comprising confirming the accuracy of injection using MR and CT imaging.

44. The method of claim 42, wherein the agent is a PFC or a colloidal metal particle.

45. The method of claim 44, wherein the agent is PFOB.

46. The method of claim 41, wherein the cell is a β islet cell transplanted into the kidney.

47. A method of labeling a cell for in vivo imaging with a labeling agent comprising:
electroporating the cell in the presence of a metal containing particle;
thereby labeling the cell with a multimodal labeling agent.

48. The method of claim 45, wherein the agent is a multimode detectable label.

49. The method of claim 48, wherein the agent is a dextran based particle.

50. The method of claim 49, wherein the particle is an iron dextran particle, a gold dextran particle, or a silver dextran particle.

51. The method of claim 47, wherein the cell is a stem cell.

52. The method of claim 51, wherein the cell is a mesenchymal stem cell.

53. A method of labeling a cell in vivo comprising:
administering to a subject a multimodal imaging agent;
thereby labeling a cell in vivo.

54. The method of claim 53, wherein the multimodal imaging agent is specifically targeted to a specific cell type.

55. The method of claim 53, wherein the labeling agent is a colloidal metal particle.

56. The method of claim 53, wherein the particle is a core-shell particle.

57. The method of claim 56, wherein the shell of the core-shell particle is derivatized with functional groups for the conjugation of a bioactive molecule.

58. The method of claim 57, wherein the bioactive molecule is a peptide or polypeptide.

59. The method of claim 58, wherein the peptide or polypeptide is an antibody or fragment thereof.

60. The method of claim 59 wherein the antibody or fragment thereof targets the labeling agent to a specific cell type.

61. The method of claim 60, wherein the cell type is a cancer cell.

62. An ex vivo-labeled cell for multimodal in vivo imaging produced by the method of any one of claims 1-49.

63. A method of locating a cell comprising a multimode-detectable labeling agent in a subject comprising:
obtaining two or more images of the subject or a portion thereof;
overlaying the images;
analyzing the images to determine the location of the cell in the subject.

64. The method of claim 63, wherein the cell is the cell of claim 62.

65. The method of claim 63, wherein the images are selected from X-ray, CT, ultrasound, Raman, and magnetic resonance images.

66. The method of claim 63, wherein the overlaying and analysis step is preformed using a computer program.

67. A method of measuring the presence of a cell labeled with a fluorescent contrast agent comprising;
labeling a cell with a fluorescent agent;
irradiating a tissue comprising the cell with radiation;
detecting a fluorescence emission spectrum of the fluorescent agent;
identifying a chromophore in the tissue from the spectrum.

68. A method for determining if a cell contains a single or multiple contrast agents that produce a Raman spectra:
a) labeling a cell with a raman reporting contrast agent by the method of any one of claims 1-50 or by administering a contrast agent with antibody bound to the contrast agent so after systemic administration it binds to the antibody target
b) irradiating the tissue with a beam of infrared monochromatic light;
c) obtaining the infrared Raman spectrum from the labeled cell
d) comparing said infrared Raman spectrum so obtained from the labeled cells with the infrared Raman spectra correspondingly obtained from known samples of cells non containing contrast agent

69. A system for monitoring a the presence of a raman detectable agent in or on a cell using low-resolution Raman spectroscopy comprising:
a catheter having a first end and a second end with an excitation fiber extending therebetween, the excitation fiber suitable to transmit multi-mode radiation from the first end to the second end to irradiate a target region;
a multi-mode laser coupled to the first end of the excitation fiber, the laser generates multi-mode radiation for irradiating the target region to produce a Raman spectrum consisting of scattered electromagnetic radiation;
a low-resolution dispersion element positioned to receive and separate the scattered radiation into different wavelength components;
a detection array, optically aligned with the dispersion element for detecting at least some of the wavelength components of the scattered light; and
a processor for processing the data from the detector array to monitor a Raman detectable agent

70. A kit comprising the cell produced by the method of any one of claim 1-49 and instructions for use.

71. A kit comprising reagents for labeling a cell for multimode-imaging and instructions for use.

72. A kit comprising the cell of claim 62 and instructions for use.

73. A kit comprising a cyropreserved cell that is labeled with a multimode-detectable labeling agent and instructions for use.

74. A kit for comprising β islet cells comprising a multimodal-detectable label and instructions for transplanting the cell in to a subject.
